(19) United States
(12) Patent Application Publication
(10) Pub. No.: US 2018/0028267 A1
(43) Pub. Date: Feb. 1, 2018

(54) RADIO-FREQUENCY ELECTRICAL MEMBRANE BREAKDOWN FOR THE TREATMENT OF BENIGN PROSTATIC HYPERPLASIA

(71) Applicant: RFEMB Holdings, LLC, North Brandford, CT (US)

(72) Inventors: Gary M. Onik, Ft. Lauderdale, FL (US); James A. Miessau, Branford, CT (US); David G. Bostwick, Orlando, FL (US)

(21) Appl. No.: 15/548,877

(22) PCT Filed: Feb. 3, 2016
(86) PCT No.: PCT/US2016/016300

§ 371 Date: Aug. 4, 2017

Related U.S. Application Data
(60) Provisional application No. 62/111,854, filed on Feb. 4, 2015.

(51) Int. Cl. A61B 34/20; A61B 5/06; A61B 5/055; A61B 5/00; A61B 10/02; A61F 2/04; A61B 8/12; A61B 18/14; A61B 8/08

(52) U.S. Cl. CPC A61B 34/20; A61B 18/1477; A61B 5/066; A61B 5/055; A61B 8/0841; A61B 8/483; A61B 10/0241; A61B 18/1492; A61F 2/04; A61B 8/12; A61B 5/0035; A61B 2018/00577

(57) ABSTRACT

An imaging, guidance, planning and treatment system integrated into a single unit or assembly of components, and a method for using same, that can be safely and effectively deployed to treat Benign Prostatic Hyperplasia in ail medical settings, including in a physician's office or in an outpatient setting. The system utilizes the novel process of Radio-Frequency Electrical Membrane Breakdown (“EMB” or “RFEMB”) to destroy the cellular membranes of unwanted BPH tissue without damaging sensitive anatomical structures in the prostate. The system preferably comprises at least one EMB treatment probe 20, at least one ultrasound scanner, at least one trackable anesthesia needle 300, and at least one controller unit for at least partially automating the treatment process.

### CROSS-REFERENCE TO RELATED APPLICATIONS

The present application is a continuation of U.S. Provisional Patent Application No. 62/111,854, filed Feb. 4, 2015, which is a continuation-in-part of U.S. patent application Ser. No. 14/451,333, filed Aug. 4, 2014, which claims priority from U.S. Provisional Patent Application Nos. 61/912,172, filed Dec. 5, 2013, 61/861,565, filed Aug. 2, 2013, and 61/867,048, filed Aug. 17, 2013, all of which are incorporated herein by reference.

### BACKGROUND OF THE INVENTION

### 1. Field of the Invention

The present invention relates generally to medical devices and treatment methods, and more particularly, to a device and method of treating benign prostatic hyperplasia (BPH) by ablating unwanted tissue causing BPH, using applied electric fields.

### 2. Background of the Invention

Tissue ablation is another, minimally invasive method of destroying undesirable tissue in the body, and has been generally divided into thermal and non-thermal ablation technologies. Thermal ablation encompasses both the addition and removal of heat to destroy undesirable cells. Cryoablation is a well-established technique that kills cells by freezing of the extracellular compartment resulting in cell dehydration beginning at −15 C and by intracellular ice formation causing membrane rupture occurring at colder temperatures.

Heat based techniques are also well established for ablation of both cancerous and non-cancerous tissues and include radio-frequency (RF) thermal, microwave and high intensity focused ultrasound ablation which raise localized tissue temperatures well above the body's normal 37° C. These methods use various techniques to apply energy to the target cells to raise interstitial temperature. For example, RF thermal ablation uses a high frequency electric field to induce vibrations in the cell membrane that are converted to heat by friction. Cell death occurs in as little as thirty (30) seconds once the cell temperature reaches 50° C. and increases as the temperature rises. At 60° C., cell death is instantaneous. If the intracellular temperature rises to between about 60° C. and 95° C., the mechanisms involved in cell death include cellular desiccation and protein coagulation. When the intracellular temperature reaches 100° C., cellular vaporization occurs as intracellular water boils to steam. In the context of tissue ablation, cell temperatures not exceeding 50° C. are not considered clinically significant, Because cellular proteins are denatured by the heat of thermal ablation techniques, they are not available to stimulate a specific immune response. Both heat-based and cryoablation techniques suffer from the drawback that they have little or no ability to spare normal structures in the treatment zone and so can be contraindicated based on tumor location or lead to complications from collateral injury. Mapping biopsies, guided by ultrasound and augmented by information from sophisticated imaging such as MRI, can allow exact targeting of a patient's cancer to enable a targeted focal ablation method.

Non-thermal ablation techniques include electrochemotherapy and irreversible electroporation (IRE) which, although quite distinct from one another, each rely on the phenomenon of electroporation. With reference to FIG. 1, electroporation refers to the fact that the plasma membrane of a cell exposed to high voltage pulsed electric fields within certain parameters becomes temporarily permeable due to destabilization of the lipid bilayer and the formation of pores P. The cell plasma membrane consists of a lipid bilayer with a thickness t of approximately 5 nm. With reference to FIG. 2(A), the membrane acts as a non-conducting, dielectric barrier forming, in essence, a capacitor. Physiological conditions produce a natural electric potential difference due to charge separation across the membrane between the inside and outside of the cell even in the absence of an applied electric field. This resting transmembrane electric potential (V′m) ranges from 40 mv for adipose cells to 85 mv for skeletal muscle cells and 90 mv for cardiac muscle cells and can vary by cell size and ion concentration, among other things.

With continued reference to FIGS. 2(B)-2(D), exposure of a cell to an externally applied electric field E induces an additional voltage V across the membrane as long as the external field is present. The induced transmembrane voltage is proportional to the strength of the external electric field and the radius of the cell. Formation of transmembrane pores P in the membrane occurs if the cumulative resting and applied transmembrane potential exceeds the threshold voltage, which may typically be between 200 mV and 1 V. Poration of the membrane is reversible if the transmembrane potential does not exceed the critical value such that the pore area is small in relation to the total membrane surface. In such reversible electroporation, the cell membrane recovers after the applied field is removed and the cell remains viable. Above a critical transmembrane potential and with longer exposure times, potation becomes irreversible, leading to eventual cell death due an influx of extracellular ions resulting in loss of homeostasis and subsequent apoptosis. Pathology after irreversible electroporation of a cell does not show structural or cellular changes until twenty-four (24) hours after field exposure except in certain very limited tissue types. However, in all cases the mechanism of cellular destruction and death by IRE is apoptotic, which requires considerable time to pass and is not visible pathologically in a time frame to be clinically useful in determining the efficacy of IRE treatment, which is an important clinical drawback to the method.

Developed in the early 1990's, electrochemotherapy combines the physical effect of reversible cell membrane poration with administration of chemotherapy drugs such as cisplatin and bleomycin. By temporarily increasing the cell membrane permeability, uptake of non-permeant or poorly permeant chemotherapeutic drugs is greatly enhanced. After the electric field is discontinued, the pores close and the drug molecules are retained inside the target cells without significant damage to the exposed cells. This approach to chemotherapy grew out of earlier research developing electroporation as a technique for transfection of genes and DNA molecules for therapeutic effect. In this context, IRE leading to cell death was viewed as a failure in as much as the treated cells did not survive to realize the modification as intended.

IRE as an ablation method grew out of the realization that the “failure” to achieve reversible electroporation could be utilized to selectively kill undesired tissue. IRE effectively kills a predictable treatment area without the drawbacks of thermal ablation methods that destroy adjacent vascular and collagen structures. During a typical IRE treatment, one to three pairs of electrodes are placed in or around tumor. Electrical pulses carefully chosen to induce an electrical field strength above the critical transmembrane potential are delivered in groups of ten (10), usually for nine (9) cycles. Each ten-pulse cycle takes about one second, and the electrodes pause briefly before starting the next cycle. As described in U.S. Pat. No. 8,048,067 to Rubinsky, et. al and U.S. patent application Ser. No. 13/332,111 to Arena, et al., both of which are incorporated here by reference, the field strength and pulse characteristics are chosen to provide the necessary field strength for IRE but without inducing thermal effects as with RF thermal ablation.

However, the DC pulses used in currently available IRE methods and devices have characteristics that can limit their use or add risks for the patient because current methods and devices create severe muscle contraction during treatments. This is a significant disadvantage because it requires that a patient be placed and supported under general anesthesia with neuromuscular blockade in order for the procedure to be carried out, and this carries with it additional substantial inherent patient risks and costs. Moreover, since even relatively small muscular contractions can disrupt the proper placement of IRE electrodes, the efficacy of each additional pulse train used in a therapy regimen may be compromised without even being noticed during the treatment session.

What is needed is a minimally invasive tissue ablation technology that can avoid damaging healthy tissue.

It would also be advantageous to provide a system and method for carrying out this treatment in a medical setting such as a physician's office or outpatient setting under local anesthesia, using a method that does not require general anesthesia or a neuromuscular blockade.

Benign Prostatic Hyperplasia (BPH)

The prostate is a walnut-sized gland that forms part of the male reproductive system. The gland consists of several lobes, or regions, enclosed by a dense fibrous capsule. It is located between the bladder and the rectum and wraps around the urethra, the tube that carries urine out from the bladder through the penis. There are generally three glandular zones in a prostate gland: central, peripheral and transitional. The transitional zone is located right behind the place where the seminal vesicles merge with the urethra. This transitional zone tends to be predisposed to benign enlargement. The prostate gland is generally composed of smooth muscles and glandular epithelial tissue. The glandular epithelial tissue produces prostatic fluid. The smooth muscles contract during sexual climax and squeeze the prostatic fluid into the urethra as the sperm passes through the ejaculatory ducts and urethra. Prostatic fluid secreted by the prostate gland provides nutrition for ejaculated spermatozoids, increases their mobility, and improves the spermatozoids' chances for survival after ejaculation by making the environment in the vaginal canal less acidic.

The prostate reaches its normal size and weight (about 20 grams) soon after puberty. The size and weight of the prostate typically remain stable until the individual reaches his mid-forties. At this age, the prostate typically begins to enlarge through a process of excessive cell proliferation, called benign prostatic hyperplasia (BPH). This overgrowth can occur in both smooth muscle and glandular epithelial tissues and has been attributed to a number of different causes, including hormones and growth factors as well as generally to the aging process.

Benign prostate hyperplasia can cause distressing urination symptoms. As the disease progresses, the dense capsule surrounding the enlarging prostate prevents it from further expansion outward and forces the prostate to press against the urethra, partially obstructing the urine flow. The tension in the smooth muscles of the prostate also increases, which causes further compression of the urethra and reduction of the urine flow. Some symptoms of BPH stem from the gradual loss of bladder function, leading to an incomplete emptying of the bladder. The symptoms can include straining to urinate, a weak or intermittent stream, an increased frequency of urination, pain during urination, and incontinence, the involuntary loss of urine following an uncontrollable sense of urgency. These symptoms alone can negatively affect the quality of life of affected men. Left untreated, BPH can cause even more severe complications, such as urinary tract infection, acute urinary retention, and uremia.

Before age 40, only 10% of men have BPH; but by age 80, about 80% have signs of this condition. BPH is the most mon non-cancerous form of cell growth in men. About 14 million men in United States have BPH, and about 375,000 new patients are diagnosed every year.

For many years, researchers have tried to find medications to shrink the prostate or at least stop its growth. Between 1992 and 1997 the FDA approved font drugs to treatment of BPH: finasteride, terazosin, tamsulosin, and doxazosin. Finasteride (Proscar) inhibits production of the hormone DHT. DHT is one of the hormones that have been found to be involved in prostate enlargement. Treatment with Finasteride has been shown to actually shrink the prostate in some men. Terazosin (Hytrin), doxazosin (Cardura), and tamsulosin belong to the class of drugs known as alpha-blockers. Alpha-blockers act by relaxing the smooth muscle of the prostate and bladder to improve urine flow and reduce bladder outlet obstruction. In men with severe symptoms, though, these medications are not curative. They can delay but not prevent the eventual need for surgery.

Regardless of the efficacy of any drug treatment, the long term exposure to xenobiotic compounds may produce additional unwanted side effects that are not realized until years after treatment. Accordingly, a need exists for an apparatus and method for the treatment of BPH that does not require the introduction of xenobiotic compounds.

For men with the most severe symptoms, surgery is generally considered to be the best long-term solution. There are several surgical procedures that have been developed for relieving symptoms of BPH. However, all of them are very morbid, require a long hospital stay, generally require the use of general anesthesia, suffer from significant side effects, and have possible complications.

In recent years, a number of procedures have been introduced that are less invasive than surgery. One such procedure is the transurethral microwave thermal therapy described in U.S. Pat. No. 5,575,511 to Reid et al. In transurethral microwave thermal therapy, a Foley-type catheter containing a microwave antenna is placed within the urethra. The microwave antenna positioned adjacent to the transitional zone of the prostate, where BPH is located, allows selective heating of the prostate. Maintaining the temperature above 45° C. during the approximately one-hour session leads to necrosis of the tissues and subsequent'reabsorption of necrotic tissue by the body.

Another recently developed non-invasive technique is transurethral needle ablation (TUNA). TUNA is described in U.S. Pat. No. 6,241,702 to Lundquist et al. TUNA uses low level radio frequency (RF) energy to heat the prostate. Using TUNA, two separate needles are inserted into prostate through the urethra. Several watts of RF energy is applied to each needle to cause thermal necrosis of the prostate cells around the needles. Application of this treatment to several sites of the prostate typically results in sufficient necrosis to relieve symptoms of the BPH.

While generally successful, the microwave and RF therapies are relatively long procedures. Also, because of the poor temperature control of the heated volume, the volume of removed tissue is often not sufficient for the long term relief of the symptoms and/or the healthy tissue of the urethra is damaged. A damaged urethra is capable of restoring itself, but the healing is a long morbid process accompanied by sloughing of the necrotic tissue into urethra and excreting it during urination. Therefore, a need exists for a minimally invasive therapy for treatment of BPH that requires shorter treatment times and is less morbid than existing therapies.

It has been suggested that IRE might be a useful modality for the treatment of BPH. However, as described above, IRE suffers from several drawbacks that limit its potential effectiveness as a BPH treatment, such as severe muscle contraction during treatment. In addition, it has been shown that sparking occurs at the junction of the insulation with the exposed portion of the IRE electrode. This sparking can cause barotrauma to the tissues with unwanted damage, thus possibly causing complications irrespective of the mechanism of action of IRE itself.

Thus, the ability to create a BPH therapy through methods that do not have the inherent limitations of either IRE or other thermal methods would be an important and substantial advancement in the treatment of BPH.

In addition, an ablation method that can be accurately targeted at previously identified BPH tissue, and that does not require general anesthesia and neuromuscular blockade, that also spares tissue structure and does not have sparking which can cause potential unwanted damage, would provide a dramatic new treatment option for patients, and form the basis for an office based procedure for the treatment of BPH.

### SUMMARY OF THE INVENTION

It is, therefore, an object of the present invention to provide a method for the treatment of Benign Prostatic Hyperplasia (BPH) in an outpatient or doctor's office setting via tissue ablation using electrical pulses which causes immediate cell death through the mechanism of complete break down of the membrane of the BPH cell.

It is another object of the present invention to provide such a treatment method that does not require the administration of general anesthesia or a neuromuscular blockade to the patient.

The present invention is an imaging, guidance, planning and treatment system integrated into a single unit or assembly of components, and a method for using same, that can be safely and effectively deployed to treat BPH in all medical settings, including in a physician's office or in an outpatient setting. The system utilizes the novel process of Radio-Frequency Electrical Membrane Breakdown (“EMB” “RFEMB”) to destroy the cellular membranes of unwanted BPH tissue without damage to the surround vital structures and tissue.

The use of EMB to achieve focal ablation of unwanted tissue while preserving vital nerves, vessels and other tissue structures, among other capabilities is disclosed in U.S. patent application Ser. No. 14/451,333 and International Patent Application No. PCT/US14/68774, which are both fully incorporated herein by reference.

EMB is the application of an external oscillating electric field to cause vibration and flexing of the cell membrane, which results in a dramatic and immediate mechanical tearing, disintegration and/or rupturing of the cell membrane. Unlike the IRE process, in which nano-pores are created in the cell membrane but through which little or no content of the cell is released, EMB completely tears open the cell membrane such that the entire contents of the cell are expelled into the extracellular fluid, and internal components of the cell membrane itself are exposed. EMB achieves this effect by applying specifically configured electric field profiles, comprising significantly higher energy levels (as much as 100 times greater) as compared to the IRE process, to directly and completely disintegrate the cell membrane rather than to electroporate the cell membrane. Such electric field profiles are not possible using currently available IRE equipment and protocols. The inability of current IRE methods and energy protocols to deliver the energy necessary to cause EMB explains why IRE treated specimens have never shown the pathologic characteristics of EMB treated specimens, and is a critical reason why EMB had not until now been recognized as an alternative method of cell destruction.

The system according to the present invention comprises a software and hardware system, and method for using the same, for detecting and measuring a mass of unwanted BPH tissue in the prostate of a patient, for designing an EMB treatment protocol to ablate said unwanted BPH tissue mass, and for applying said EMB treatment protocol in an outpatient or doctor's office setting. The system includes an EMB pulse generator 16, one or more EMB treatment probes 20, and one or more temperature probes 22. The system further employs a software-hardware controller unit (SHCU) operatively connected to said generator 16, probes 20, ultrasound scanner 500 and temperature probe(s) 22, along with one or more optional devices such as trackable anesthesia needles 300, endoscopic imaging devices, ultrasound scanners, and/or other imaging devices or energy sources, and operating software for controlling the operation of each of these hardware devices.

### BRIEF DESCRIPTION OF THE DRAWINGS

FIG. 1 is a diagram of a cell membrane pore.

FIG. 2 is a diagram of cell membrane pore formation by a prior art method.

FIG. 3 is a schematic diagram of the software and hardware system according to the present invention.

FIG. 4A is a comparison of a prior art charge reversal with an instant charge reversal according to the present invention.

FIG. 4B is a square wave from instant charge reversal pulse according to the present invention.

FIG. 5 is a diagram of the forces imposed on a cell membrane as a function of electric field pulse width according to the present invention.

FIG. 6 is a diagram of a prior art failure to deliver prescribed pulses due to excess current.

FIG. 7A is a schematic diagram depicting a US scan of a suspect tissue mass.

FIG. 7B is a schematic diagram depicting the results of a 3D Fused Image of a suspect tissue mass.

FIG. 8 is a schematic diagram depicting the target treatment area and Predicted Ablation Zone relative to a therapeutic EMB treatment probe 20 prior to delivering treatment.

FIG. 9 is a schematic diagram of a pulse generation and delivery system for application of the method o the present invention.

FIG. 10 is a diagram of the parameters of a partial pulse train according to the present invention.

FIG. 11 is a schematic diagram depicting the target treatment area and Predicted Ablation Zone relative to a therapeutic EMB treatment probe 20 at the start of treatment delivery.

FIG. 12A is a schematic diagram of a therapeutic EMB treatment probe 20 according to one embodiment of the present invention.

FIG. 12B is a composite schematic diagram (1, 2 and 3) of the therapeutic EMB treatment probe 20 of FIG. 12A showing insulating sheath 23 in various stages of retraction.

FIG. 12C is a composite schematic diagram (1 and 2) of a therapeutic EMB treatment probe 20 according to another embodiment of the present invention.

FIG. 12D is a composite schematic diagram (1 and 2) of the therapeutic EMB treatment probe 20 of FIG. 12C showing insulating sheath 23 in various stages of retraction.

FIG. 13 is a schematic diagram of an EMB treatment probe 20 comprising a sharp, rigid needle according to another embodiment of the present invention, depicting how two needles may be used, one with a positive and cane with a negative electrode.

FIG. 14 is a schematic diagram of the enhanced trackable anesthesia needle 300 according to the present invention.

FIG. 15 is as schematic diagram depicting the positioning of a catheter-type therapeutic EMB treatment probe 20 according to an embodiment of the present invention proximate the treatment area 2.

FIG. 16 is a schematic diagram depicting the positioning of a catheter-type therapeutic EMB treatment probe 20 comprising a thermocouple 7 according to another embodiment of the present invention proximate the treatment area 2.

FIG. 17 is a schematic diagram depicting the positioning of a catheter-type therapeutic EMB treatment probe 20 comprising a side port 8 for exposure of needle 9 according to another embodiment of the present invention proximate the treatment area 2.

FIG. 18 is a schematic diagram depicting the positioning of a rigid needle-type therapeutic EMB treatment probe 20 comprising a unipolar electrode 11 according to another embodiment of the present invention proximate the treatment area 2, wherein a remote electrode 15 is placed near another area of the patient's body.

FIG. 19 is a schematic diagram depicting the positioning of a catheter-type therapeutic EMB treatment probe 20 comprising a side port 8 for exposure of electrode-bearing needle 17 according to another embodiment of the present invention proximate the treatment area 2.

FIG. 20 is a schematic diagram depicting the positioning of a catheter-type therapeutic EMB treatment probe 20 comprising EM sensor 26 according to another embodiment of the present invention inside the urethra.

FIG. 21 is a schematic diagram depicting the positioning of a catheter-type therapeutic EMB treatment probe 20 comprising an expandable stabilizing balloon 27 according to another embodiment of the present invention inside the urethra.

FIG. 22 is a schematic diagram depicting the positioning of a catheter-type therapeutic EMB treatment probe 20 comprising an expandable electrode-bearing balloon 27 according to another embodiment of the present invention inside the urethra.

FIG. 23 is a schematic diagram depicting the positioning of a catheter-type therapeutic EMB treatment probe 20 comprising an electrode-bearing sheath 23 according to another embodiment of the present invention inside the urethra.

FIG. 24 is a schematic diagram depicting the use of two therapeutic EMB treatment probes 20 for delivery of EMB treatment.

FIG. 25 is a schematic diagram depicting the positioning of a rigid needle-type therapeutic EMB treatment probe 20 comprising a bipolar electrode.

FIG. 26 is a composite (A & B) schematic diagram depicting the positioning of a catheter-type therapeutic EMB treatment probe 20 comprising an inflatable stent 19 according to another embodiment of the present invention inside the urethra.

FIG. 27 is a schematic diagram depicting the positioning of a stent 19 left by catheter-type EMB treatment probe 20 inside the urethra.

FIG. 28 is a schematic diagram depicting the use of a non-electrode bearing inflatable balloon 27 with catheter-type EMB treatment probe 20 inside the bladder of a patient for the treatment of BPH.

FIG. 29 is a schematic diagram depicting two electrode-bearing needles 17 inserted through a scope for the treatment of BPH.

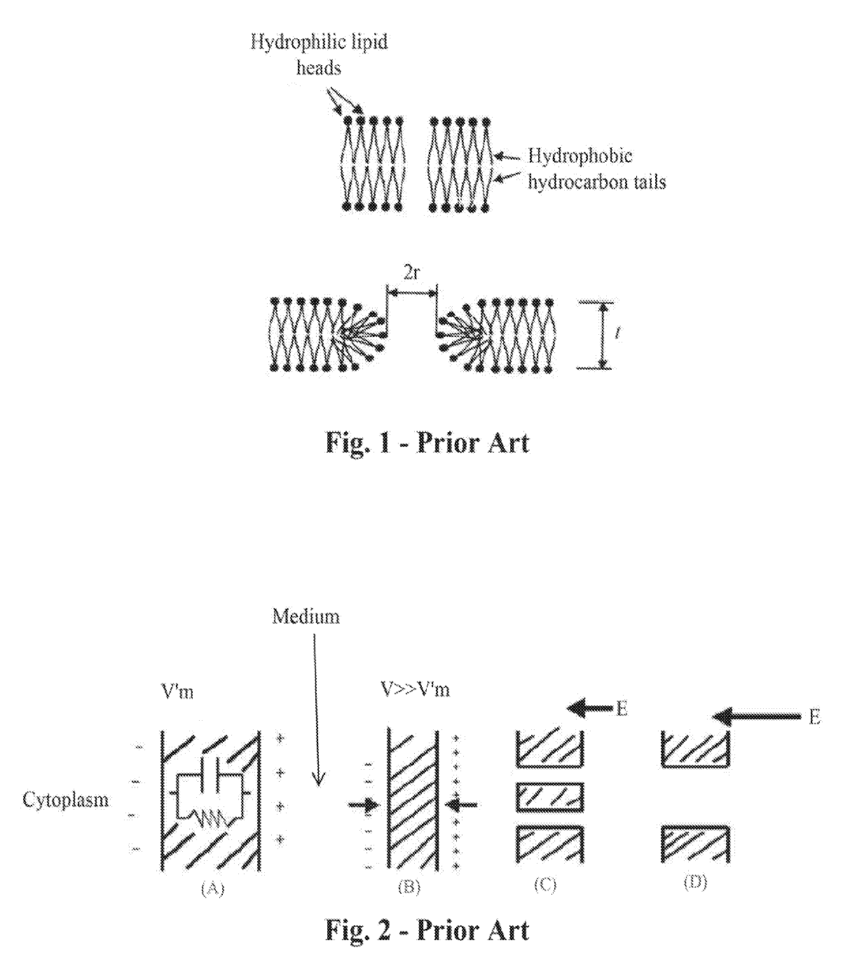

### DETAILED DESCRIPTION

In general, the software-hardware controller unit (SHCU) operating the proprietary office based BPH treatment system software according to the present invention facilitates the treatment of BPH by directing the placement of EMB treatment probe(s) 20, and, optionally, anesthesia needle(s) 300, and by delivering electric pulses designed to cause EMB within the unwanted BPH tissue to EMB treatment probe(s) 20, all while the entire process may be monitored in real time via one or more two- or three-dimensional imaging devices scans taken at strategic locations to measure the extent of BPH tissue cell death. The system is such that the treatment may be performed by a physician under the guidance of the software, or may be performed completely automatically, from the process of imaging the treatment area 2 to the process of placing one or more probes using robotic arms operatively connected to the SHCU to the process of delivering electric pulses and monitoring the results of same. Specific components of the invention will now be described in greater detail.

EMB Pulse Generator 16

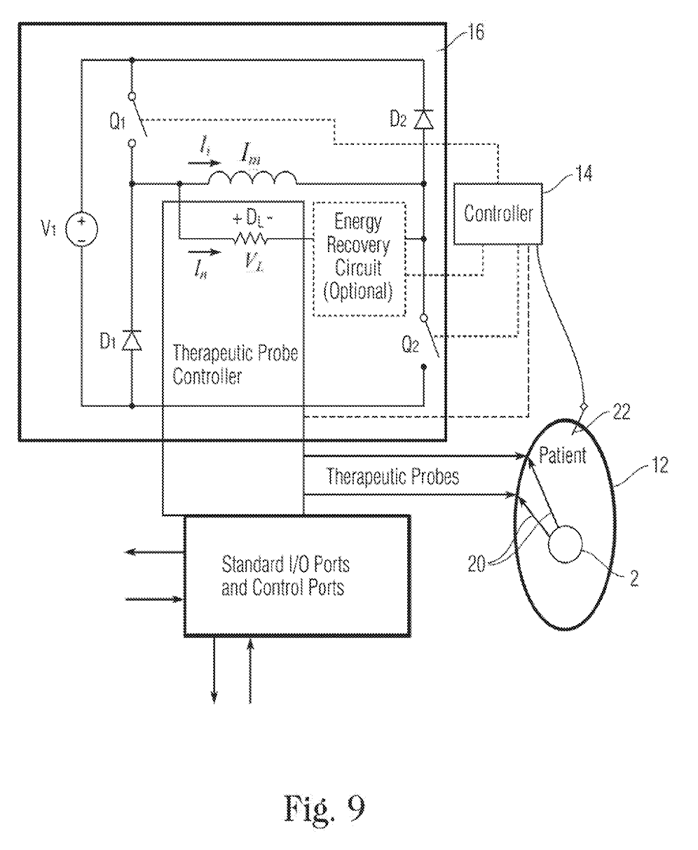

FIG. 9 is a schematic diagram of a system for generation of the electric field necessary to induce EMB of cells 2 within a patient 12. The system includes the EMB pulse generator 16 operatively coupled to Software Hardware Control Unit (SHCU) 14 for controlling generation and delivery to the EMB treatment probes 20 (two are shown) of the electrical pulses necessary to generate an appropriate electric field to achieve EMB. FIG. 9 also depicts optional onboard controller 15 which is preferably the point of interface between EMB pulse generator 16 and SHCU 14. Thus, onboard controller 15 may perform functions such as accepting triggering data from SHCU 14 for relay to pulse generator 16 and providing feedback to SHCU regarding the functioning of the pulse generator 16. The EMB treatment probes 20 (described in greater detail below) are placed in proximity to the unwanted BPH soft tissue 2 which are intended to be ablated through the process of EMB and the bipolar pulses are shaped, designed and applied to achieve that result in an optimal fashion. A temperature probe 22 may be provided for percutaneous temperature measurement and feedback to the controller of the temperature at, on or near the electrodes. The controller may preferably include an onboard digital processor and a memory and may be a general purpose computer system, programmable logic controller or similar digital logic control device. The controller is preferably configured to control the signal output characteristics of the signal generation including the voltage, frequency, shape, polarity and duration of pulses as well as the total number of pulses delivered in a pulse train and the duration of the niter pulse burst interval.

With continued reference to FIG. 9, the EMB protocol calls for a series of short and intense bi-polar electric pulses delivered from the pulse generator through one or more EMB treatment probes 20 inserted directly into, or placed around the target tissue 2. The bi-polar pulses generate an oscillating electric field between the electrodes that induce a similarly rapid and oscillating buildup of transmembrane potential across the cell membrane. The built up charge applies an oscillating and flexing force to the cellular membrane which upon reaching a critical value causes rupture oldie membrane and spillage of the cellular content. Bipolar pulses are more lethal than monopolar pulses because the pulsed electric field causes movement of charged molecules in the cell membrane and reversal in the orientation or polarity of the electric field causes a corresponding change in the direction of movement of the charged molecules and of the forces acting on the cell. The added stresses that are placed on the cell membrane by alternating changes in the movement of charged molecules create additional internal and external changes that cause indentations, crevasses, rifts and irregular sudden tears in the cell membrane causing more extensive, diverse and random damage, and disintegration of the cell membrane.

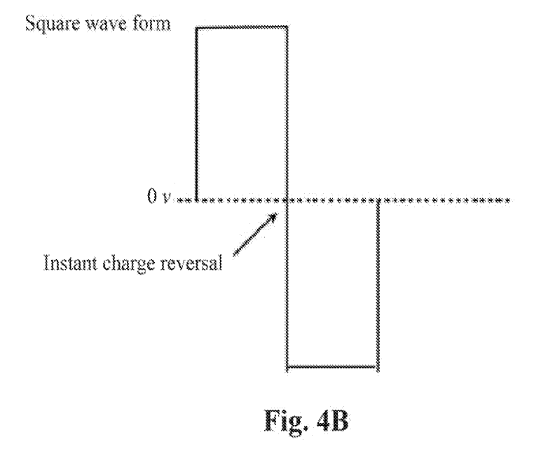

With reference to FIG. 4B, in addition to being bi-polar, the preferred embodiment of electric pulses is one for which the voltage over time traces a square wave form and is characterized by instant charge reversal pulses (ICR). A square voltage wave form is one that maintains a substantially constant volume of not less than 80% of peak voltage for the duration of the single polarity portion of the trace, except during the polarity transition. An instant charge reversal pulse is a pulse that is specifically designed to ensure that substantially no relaxation time is permitted between the positive and negative polarities of the bi-polar pulse (See FIG. 4A). That is, the polarity transition happens virtually instantaneously.

The destruction of dielectric cell membranes through the process of Electrical Membrane Breakdown is significantly more effective if the applied voltage pulse can transition from a positive to a negative polarity without delay in between. Instant charge reversal prevents rearrangement of induced surface charges resulting in a short state of tension and transient mechanical forces in the cells, the effects of which are amplified by large and abrupt force reversals. Alternating stress on the target cell that causes structural fatigue is thought to reduce the critical electric field strength required for EMB. The added structural fatigue inside and along the cell membrane results in or contributes to physical changes in the structure of the cell. These physical changes and defects appear in response to the force applied with the oscillating EMB protocol and approach dielectric membrane breakdown as the membrane position shifts in response to the oscillation, up to the point of total membrane rupture and catastrophic discharge. This can be analogized to fatigue or weakening of a material caused by progressive and localized structural damage that occurs when a material is subjected to cyclic loading, such as for example a metal paper clip that is subjected to repeated bending. The nominal maximum stress values that cause such damage may be much less than the strength of the material under ordinary conditions. The effectiveness of this waveform compared to other pulse waveforms can save up to ⅕ or ⅙ of the total energy requirement.

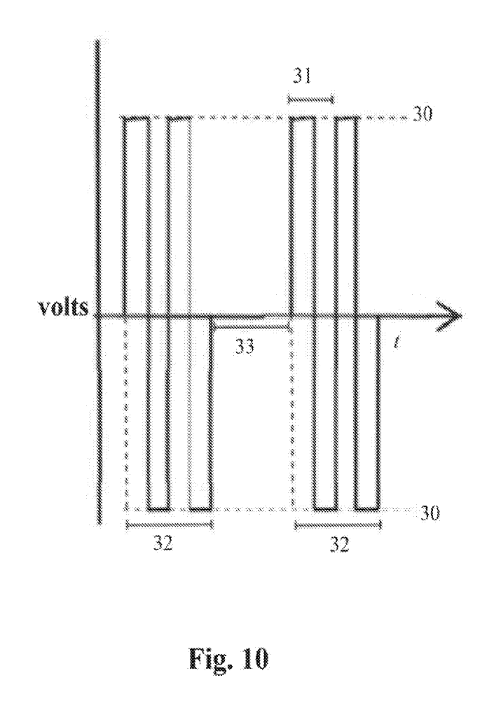

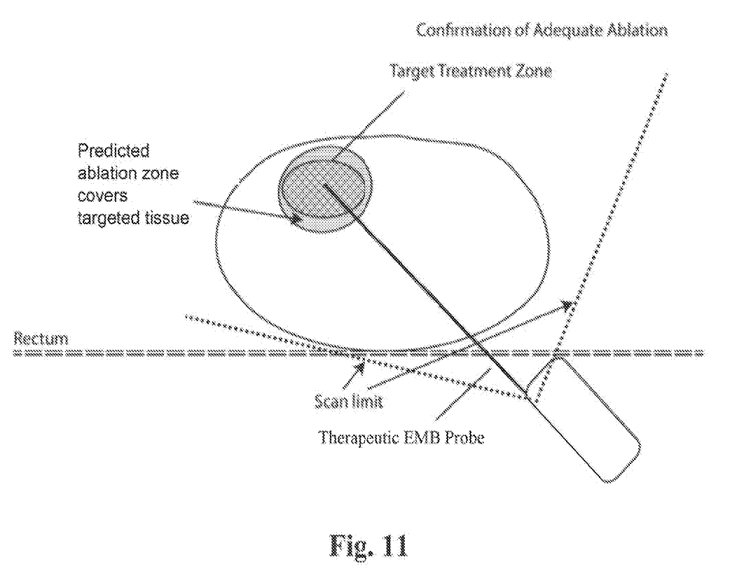

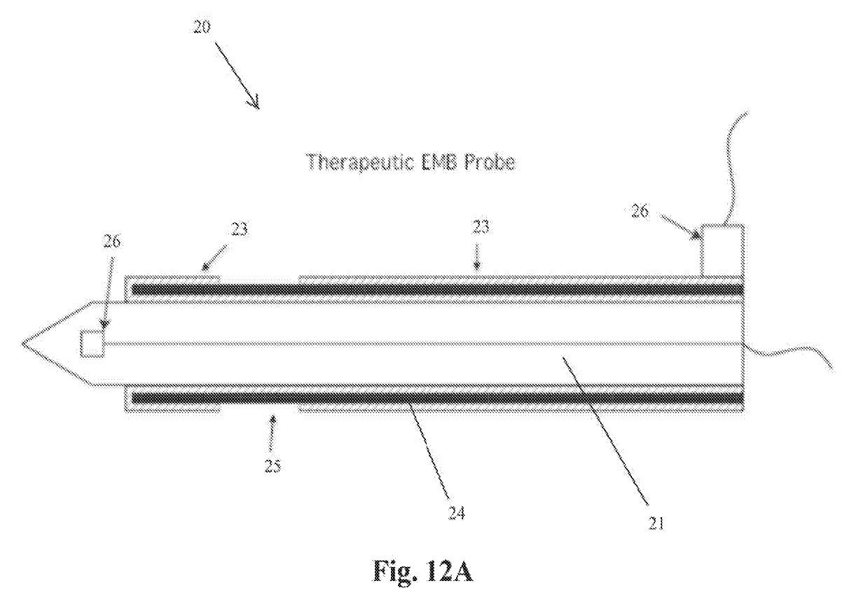

With reference to FIG. 10, another important characteristic of the applied electric field is the field strength (Volts/cm) which is a function of both the voltage 30 applied to the electrodes by the pulse generator 16 and the electrode spacing. Typical electrode spacing for a bi-polar, needle type probe might be 1 cm, while spacing between multiple needle probe electrodes can be selected by the surgeon and might typically be from 0.75 cm to 1.5 cm. A pulse generator for application of the present invention is capable of delivering up to a 10 kV potential. The actual applied field strength will vary over the course of a treatment to control circuit amperage which is the controlling factor in heat generation, and patient safety (preventing large unanticipated current flows as the tissue impedance falls during a treatment). Where voltage and thus field strength is limited by heating concerns, the duration of the treatment cycle may be extended to compensate for the diminished charge accumulation. Absent thermal considerations, a preferred field strength for EMB is in the range of 1,500 V/cm to 10,000 V/cm.

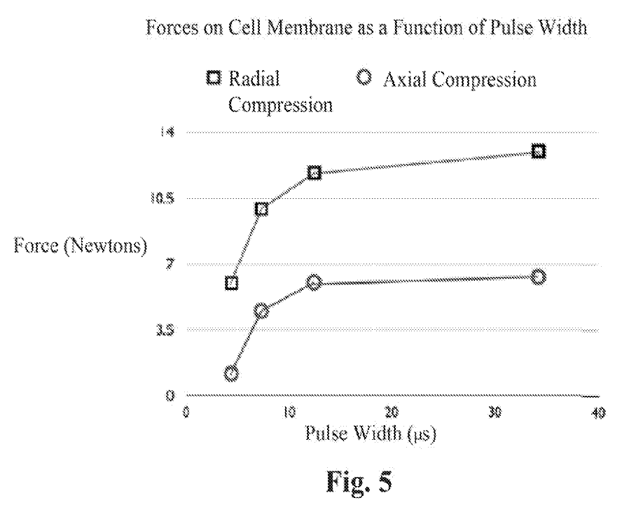

With continued reference to FIG. 10, the frequency 31 of the electric signal supplied to the EMB treatment probes 20, and thus of the field polarity oscillations of the resulting electric field, influences the total energy imparted on the subject tissue and thus the efficacy of the treatment but are less critical than other characteristics. A preferred signal frequency is from 14.2 kHz to less than 500 kHz. The lower frequency bound imparts the maximum energy per cycle below which no further incremental energy deposition is achieved. With reference to FIG. 5, the upper frequency limit is set based on the observation that above 500 kHz, the polarity oscillations are too short to develop enough motive force on the cell membrane to induce the desired cell membrane distortion and movement. More specifically, at 500 kHz the duration of a single full cycle is 2 μs of which half is of positive polarity and half negative. When the duration of a single polarity approaches 1 μs there is insufficient time for charge to accumulate and motive force to develop on the membrane. Consequently, membrane movement is reduced or eliminated and EMB does not occur. In a more preferred embodiment the signal frequency is from 100 kHz to 450 kHz. Here the lower bound is determined by a desire to avoid the need for anesthesia or neuromuscular-blocking drugs to limit or avoid the muscle contraction stimulating effects of electrical signals applied to the body. The upper bound in this more preferred embodiment is suggested by the frequency of radiofrequency thermal ablation equipment already approved by the FDA, has been deemed safe for therapeutic use in medical patients.

In addition, the energy profiles that are used to create EMB also avoid potentially serious patient risks from interference with cardiac sinus rhythm, as well as localized barotrauma, which can occur with other therapies.

EMB Treatment Probes 20

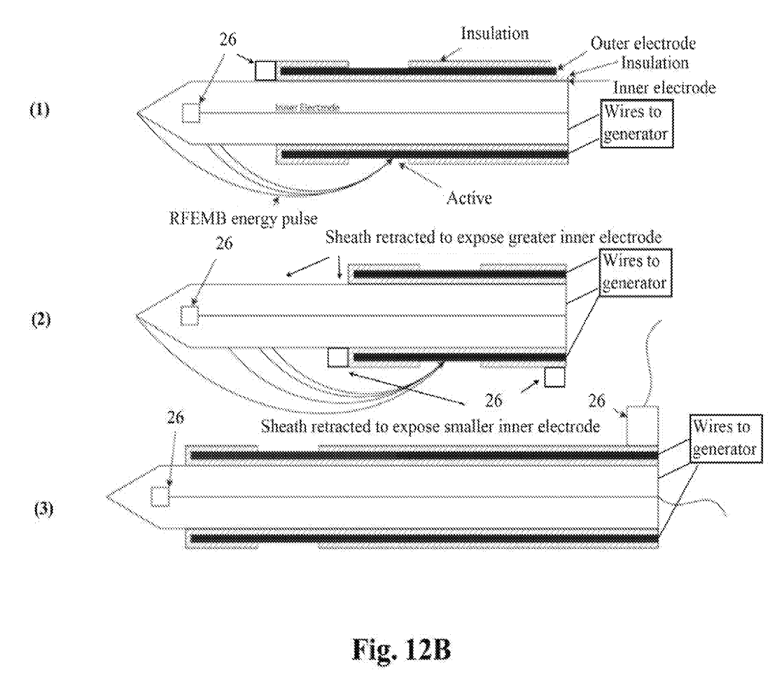

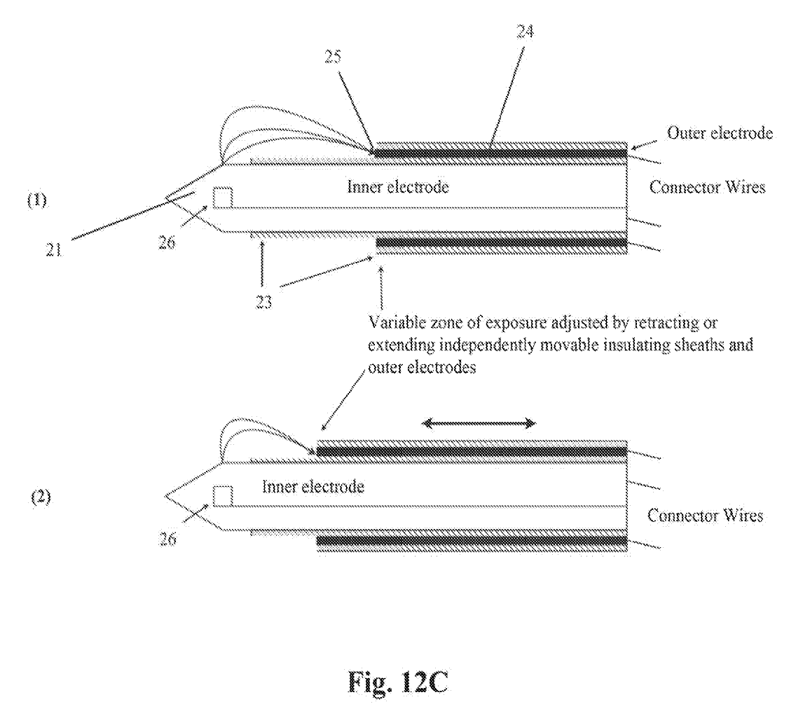

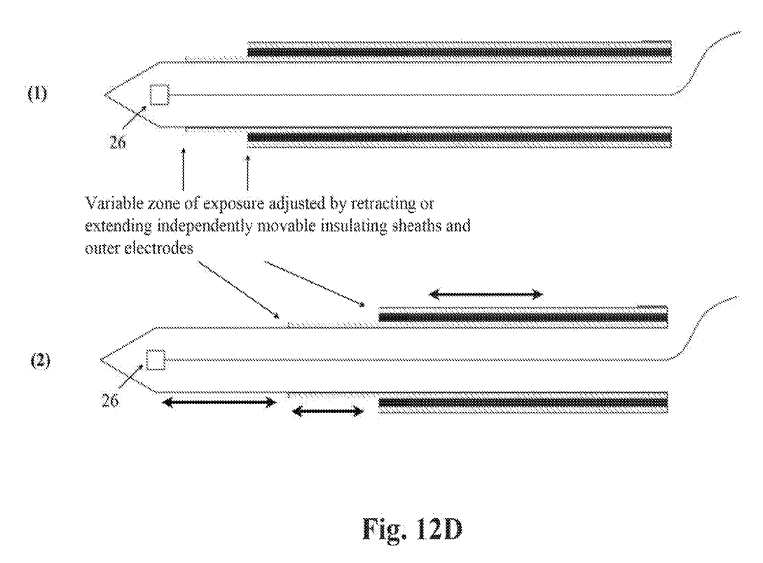

FIGS. 12A-12B depict a first embodiment of a therapeutic EMB treatment probe 20. The core (or inner electrode) 21 of EMB treatment probe 20 is preferably a needle of gage 17-22 with a length of 5-25 cm, and may be solid or hollow. Core 21 is preferably made of an electrically conductive material, such as stainless steel, and may additionally comprise one or more coatings of another conductive material, such as copper or gold, on the surface thereof. As shown in FIGS. 12A-12D, in the instant embodiment, the core 21 of treatment probe 20 has a pointed tip, wherein the pointed shape may be a 3-sided trocar point or a beveled point; however, in other embodiments, the tip may be rounded or flat. Treatment probe 20 further comprises an outer electrode 24 covering core 21 on at least one side. In a preferred embodiment, outer electrode 24 is also a cylindrical member completely surrounding the diameter of core 21. An insulating sheath 23, made of an inert material compatible with bodily tissue, such as Teflon® or Mylar®, is disposed around the exterior of core 21 and isolates core 21 from outer electrode 24. In this preferred embodiment, insulating sheath 23 is also a cylindrical body surrounding the entire diameter core 21 and completely encapsulating outer electrode 24 except at active area 25, where outer electrode 24 is exposed directly to the treatment area 2. In an alternate embodiment, shown in FIGS. 12C-12D, insulating sheath 23 comprises two solid cylindrical sheaths wherein the outer sheath completely encapsulates the lateral area of outer electrode 24 and only the distal end of outer electrode 24 is exposed to the treatment area 2 as active area 25. Insulating sheath 23 and outer electrode 24 are preferably movable as a unit along a lateral dimension of core 21 so that the surface area of core 21 that is exposed to the treatment area 2 is adjustable, thus changing the size of the lesion created by the EMB pulses. FIGS. 12B(3) and 12C(2) depict insulating sheath 23 and outer electrode 24 advanced towards the pointed tip of core 21, defining a relatively small treatment area 2, while FIGS. 12B(2) and 12C(1) depict insulating sheath 23 and outer electrode 24 retracted to define a relatively large treatment area. Electromagnetic (EM) sensors 26 on both core 21 and insulating sheath 23/outer electrode 24 member send information to the Software Hardware Controller Unit (SHCU) for determining the relative positions of these two elements and thus the size of the treatment area 2, preferably in real time. EM sensors 26 may be a passive EM tracking sensor/field generator, such as the EM tracking sensor manufactured by Traxtal Inc. Alternatively, instead of utilizing EM sensors, EMB treatment probes 20 may be tracked in real time and guided using endoscopy, ultrasound or other imaging means known in the art.

One means for enabling the relative movement between core 21 and insulating sheath 23/outer electrode 24 member is to attach insulating sheath 23/outer electrode 24 member to a fixed member (i.e., a handle) at a distal end of probe 20 opposite the tip of core 21 by a screw mechanism, the turning of which would advance and retract the insulating sheath 23/outer electrode 24 member along the body of the core 21. Other means for achieving this functionality of EMB treatment probe 20 are known in the art.

One of conductive elements 21, 24 comprises a positive electrode, while the other comprises a negative electrode. Both core 21 and outer electrode 24 are connected to the EMB pulse generator 20 through insulated conductive wires, and which are capable of delivering therapeutic EMB pulsed radio frequency energy or biphasic pulsed electrical energy under sufficient conditions and with sufficient treatment parameters to achieve the destruction and disintegration of the membranes of unwanted BPH tissue, through the process of EMB, as described in more detail above. The insulated connection wires may either be contained within the interior of EMB treatment probes 20 or on the surface thereof. However, EMB treatment probes 20 may also be designed to deliver thermal radio frequency energy treatment, if desired, as a complement to or instead of EMB treatment.

In another embodiment, EMB treatment probes 20 take the form of at least one therapeutic catheter-type probe 20. Catheter-type probes 20 are preferably of the flexible catheter type known in the art and having one or more central lumens to, among other things, allow probe 20 to be placed over a guide wire for ease of insertion and/or placement of probe 20 within a cavity 400 of the human body according to the Seldinger technique. A catheter for this purpose may be a Foley-type catheter, sized between 10 French to 20 French and made of silicone, latex or any other biocompatible, flexible material. Such a catheter is capable of being placed to deliver EMB pulses from an untraurethral location for the treatment of BPH.

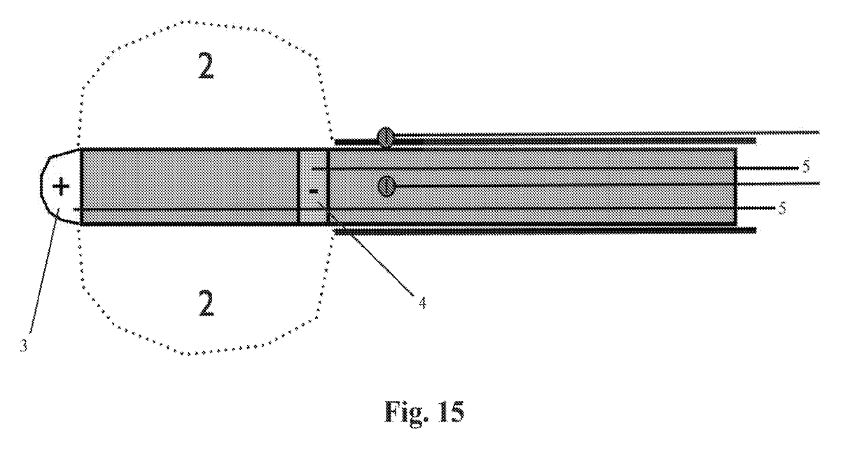

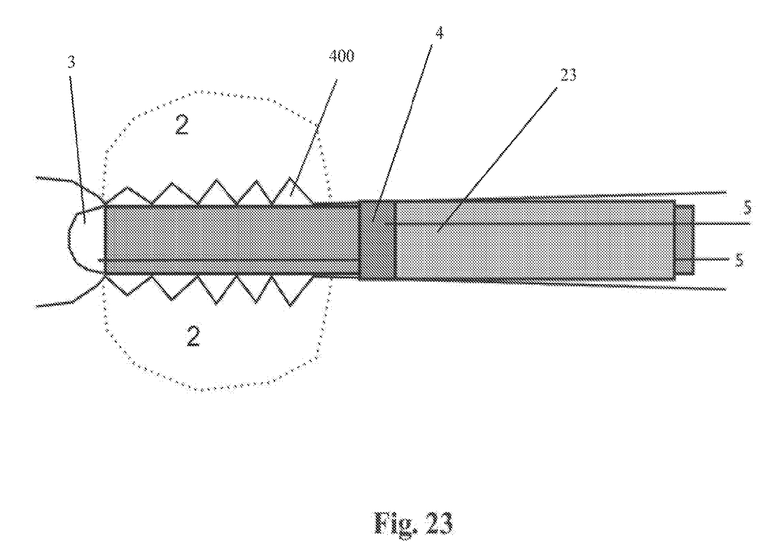

In a preferred embodiment, illustrated in FIG. 15, catheter-type probes 20 comprise one positive 3 and one negative 4 electrode disposed on an outer surface of probe 20 and spaced apart by a distance along the longitudinal axis of probe 20 such that current sufficient to deliver the EMB pulses described herein may be generated between the electrodes 3, 4. The spacing between positive 3 and negative 4 electrodes may vary by design preference, wherein a larger distance between electrodes 3, 4 provides a larger treatment area 2. FIG. 15 depicts electrodes 3, 4 on an outer surface of probe 20; alternatively, electrodes 3, 4 are integral to the surface of probe 20. In yet another embodiment, as shown in FIG. 23, one of electrodes 3, 4 (negative electrode 4 as shown in FIG. 23) may be placed on the end of an insulated sheath 23 that either partially or fully surrounds probe 20 along a radial axis thereof and is movable along a longitudinal axis of probe 20 relative to the tip thereof (on which positive electrode 3 is located as shown in FIG. 23) to provide even further customizability with respect to the distance between electrodes 3, 4 and thus the size of treatment area 2. By moving probe 20 relative to sheath 23, various distances between the electrodes can be accomplished, thus changing the size and shape of the treatment zone (see FIG. 23). Insulating sheath 23 is preferably made of an inert material compatible with bodily tissue, such as Teflon® or Mylar®. One means for enabling the relative movement between probe 20 and insulating sheath 23 is to attach insulating sheath 23 to a fixed member (i.e., a handle) at a distal end of probe 20 opposite the tip of probe 20 by a screw mechanism, the turning of which would advance and retract the insulating sheath 23 along the body of the probe 20. Other means for achieving this functionality of EMB treatment probe 20 are known in the art.

Without limitation, electrodes 3, 4 on catheter-type probes 20 may be flat (i.e., formed on only a single side of probe 20), cylindrical and surrounding probe 20 around an axis thereof, etc. Electrodes 3, 4 are made of an electrically conductive material. Electrodes 3, 4 may be operatively connected to EMB pulse generator 16 via one or more insulated wires 5 for the delivery of EMB pulses from generator 16 to the treatment area 2. Connection wires 5 may either be intraluminal to the catheter probe 20 or extra-luminal on the surface of catheter probe 20.

Electrical membrane breakdown, unlike IRE or other thermal ablation techniques, causes immediate spillage of all intracellular components of the ruptured cells into an extracellular space and exposes the internal constituent parts of the cell membrane to the extracellular space.

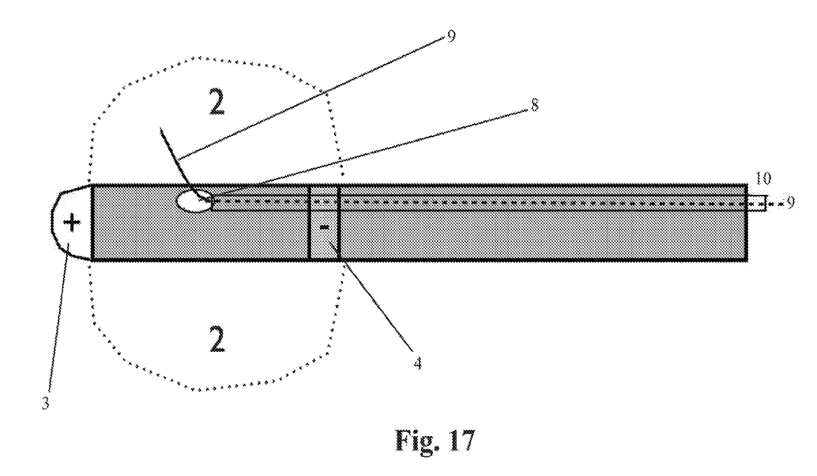

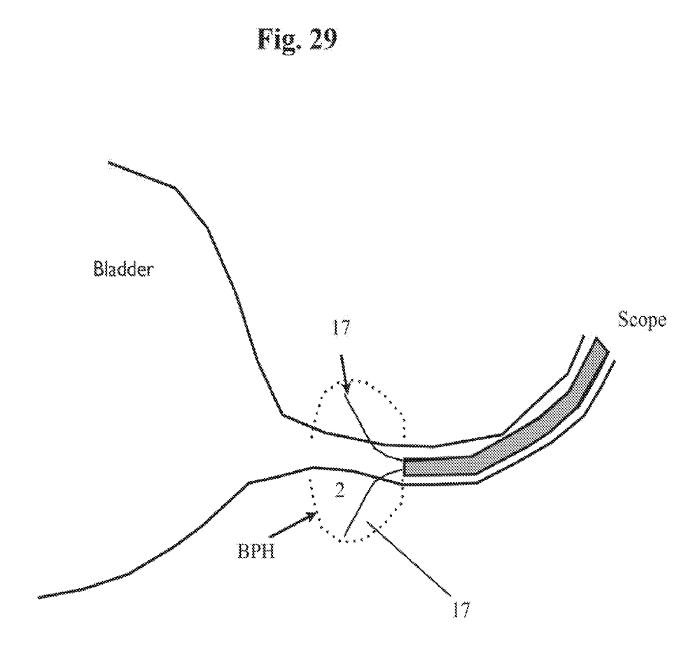

Thus, the catheter-type probe 20 according to the present invention may have a hollow interior defined by an inner lumen 10 of sufficient diameter to accommodate a spinal needle 9 of one or more standard gauges to be inserted there through for the injection of any beneficial medications or drugs into the lesion formed by EMB treatment to enhance the of said treatment (see FIG. 17). In a preferred embodiment, as shown in FIG. 17, interior lumen 10 terminates proximate an opening 8 in the side of probe 20 to allow needle 9 to exit probe 20 to access treatment area 2 for delivery of the drugs. In an alternative embodiment, shown in FIG. 29, interior lumen 10 may terminate, and one or more needle(s) 9 may exit, with an opening at distal end of probe 20. Alternatively, the inner lumen 10 may be sized to allow for the injection of biochemical or biophysical nano-materials there through into the EMB lesion to enhance the efficacy of the local ablative effect, or to allow injection of reparative growth stimulating drugs, chemicals or materials. A lumen 10 of the type described herein may also advantageously allow the collection and removal of tissue or intra-cellular components from the treatment area 2 or nearby vicinity, for examination or testing whether before, during or after treatment.

It will also be understood that, instead of a EM treatment probe having a lumen 10 capable of providing a delivery path for treatment enhancing drugs, such drugs may be administered by any means, including without limitation, intravenously, orally or intramuscularly and may further be injected directly into or adjacent to the target BPH issue immediately before or after applying the EMB electric field.

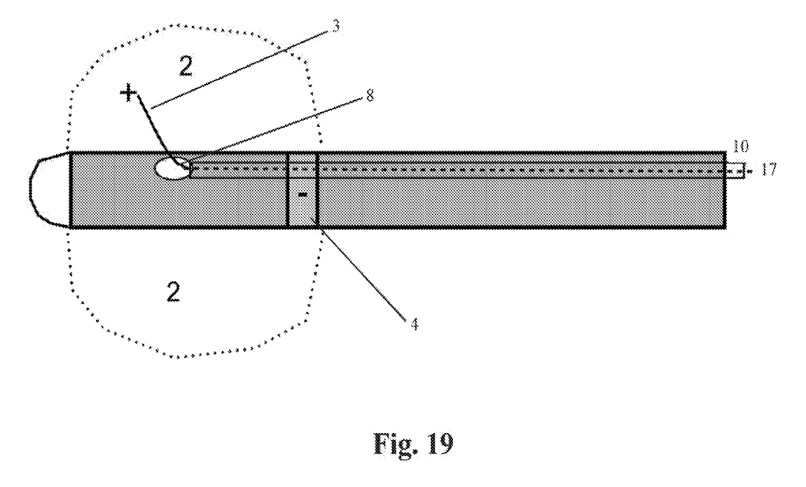

In an alternative embodiment of EMB treatment probes 20, one of either the positive (+) 3 or negative (−) 4 electrodes is on an outer surface of EMB treatment probe 20, while the other polarity of electrode is placed on the tip of a curved, electrode-bearing needle 17 inserted through lumen 10 (see FIG. 19).

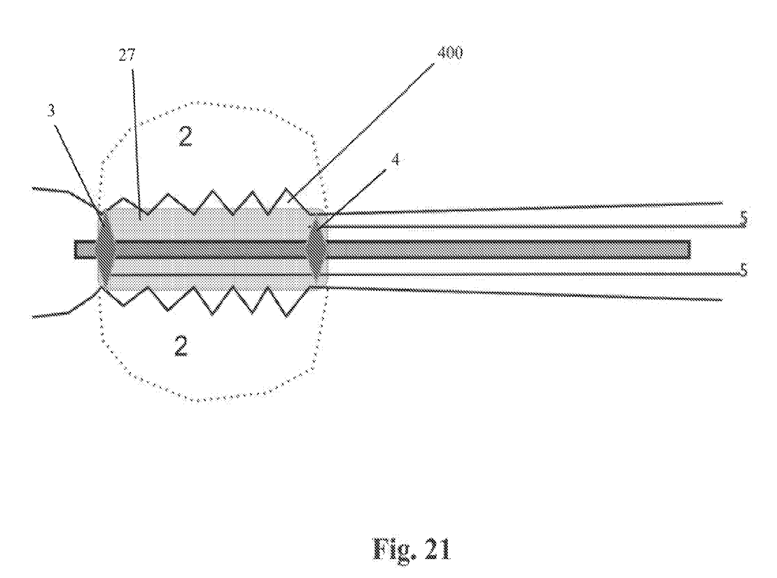

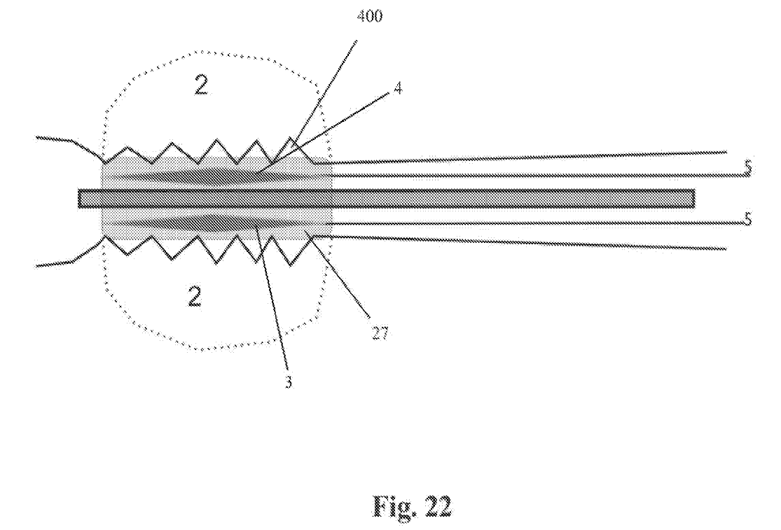

In another embodiment of EMB treatment probes 20, unipolar or bipolar electrodes ate placed on an expandable balloon 27, the inflation of which may be controlled by the SHCU via a pneumatic motor or air pump, etc. In this embodiment, when the balloon 27 is placed inside the urethra (proximate a designated treatment area 2) and inflated, the electrodes on the surface of balloon 27 are forced against the wall of the cavity 400 to provide a path for current to flow between the positive and negative electrodes (see FIG. 21). The positive and negative electrodes can have different configurations on the balloon 27, i.e., they may be arranged horizontally around the circumference of the balloon 27 as in FIG. 21, or longitudinally along the long axis of the balloon as in FIG. 22. In some embodiments, more than one each of positive and negative electrodes may be arranged on a single balloon.

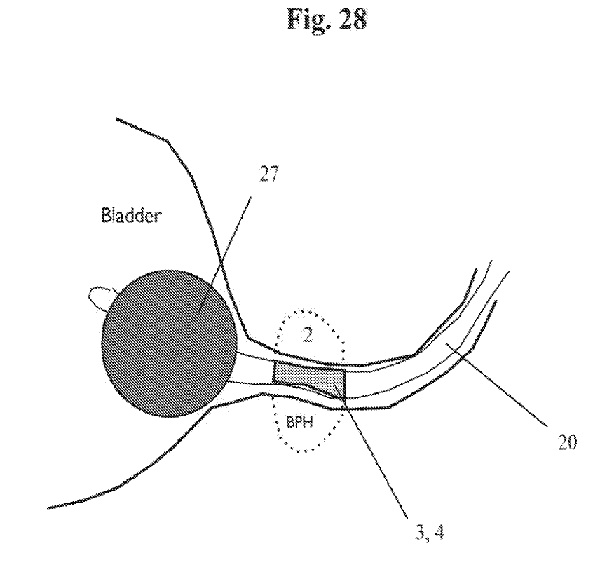

In certain embodiments, catheter-type EMB probe 20 may comprise a non-electrode-containing balloon that is otherwise of the general type described above on its distal end, such that when the balloon 27 is inflated, the catheter and EMB treatment probe 20 are anchored within the treatment area 2. Balloon 27 may anchor probe 20, inserted through the urethra for treatment of an unwanted BPH tissue mass 2 proximate the peri-urethral prostatic tissue, by a friction fit of balloon 27 in the bladder neck, as shown in FIG. 28.

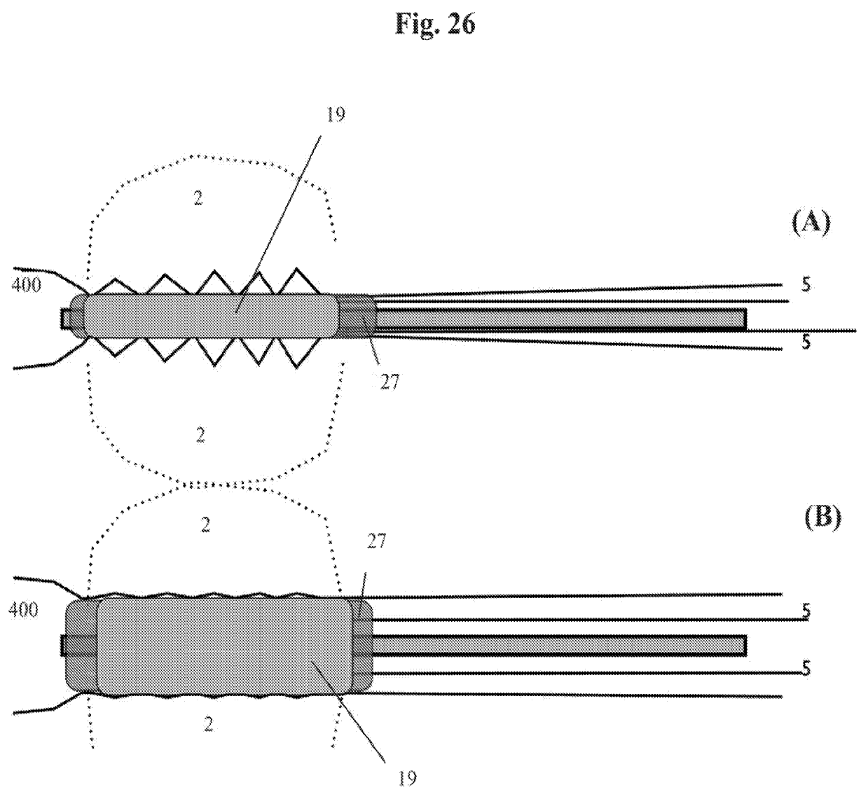

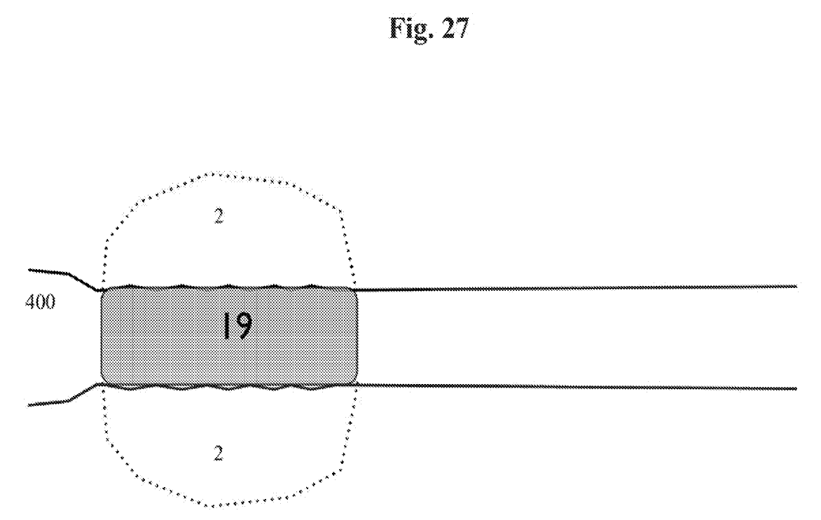

In yet another embodiment, EMB catheter-type probe 20 could deliver a stent 19 to the abnormal region/treatment area 2 which is associated with a narrowing causing obstruction. This configuration would allow the delivery of an EMB treatment protocol at the same time as stem 19 is used to expand a stricture in a lumen. Stent 19 may also comprise conducting and non-conducting areas which correspond to the unipolar or bipolar electrodes on EMB probe 20. An example treatment protocol would include placement of EMB probe 20 having balloon 27 with a stent 19 over the balloon 27 in its non expanded state (FIG. 26(A)), expansion of balloon 27 which in turn expands stent 19 (FIG. 26(B)), delivery of the RFEMB treatment, and removal of the EMB treatment probe 20 and balloon 27, leaving stent 19 in place in the patient (see FIG. 27).

Any of the therapeutic EMB treatment probes 20 described herein also preferably contain sensors of the type described by Laufer et al. in “Tissue Characterization Using Electrical Impedance Spectroscopy Data: A Linear Algebra Approach”, Physiol, Meas. 33 (2012) 997-1013, to investigate tissue characteristics to determine cancerous from non-cancerous tissue. Alternatively, or in addition to sensors of the type described by Laufer, EMB treatment probes 20 may contain sensors to determine cellular content spillage as necessary to quantify cell death in the treatment area 2 via EMB; one example of such a sensor is described by Miller et al. in “Integrated Carbon Fiber Electrodes Within Hollow Polymer Microneedles For Transdermal Electrochemical Sensing”, Biomicrofluidics, 2011 Mar. 30; 5(1):13415.

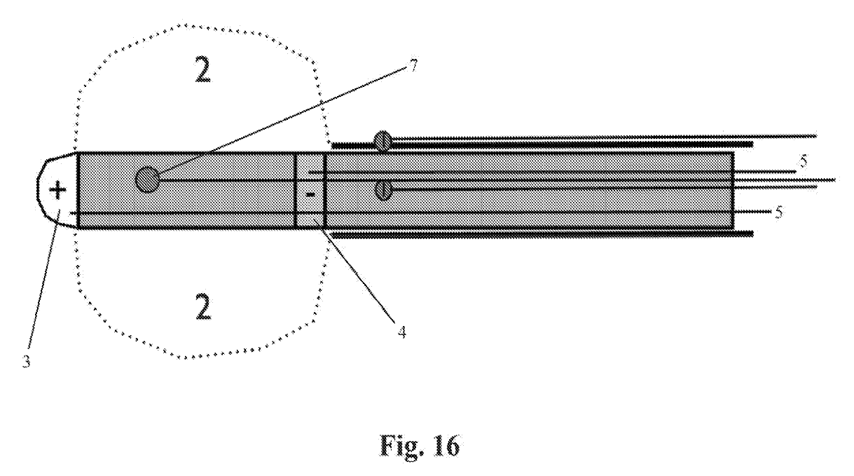

Alternatively, or in addition to the sensors described above, EMS treatment probes 20 may contain a thermocouple 7 (see FIG. 16), such as a Type K-40AWG thermocouple with Polyimide Primary/Nylon Bond Coat insulation and a temperature range of −40 to +180C, manufactured by Measurement Specialties. The lumen of the optional thermocouple 7 may be located on EMB treatment probe 20 such that the temperature at the tip of the probe can be monitored and the energy delivery to probe 20 modified to maintain a desired temperature at the tip of probe 20.

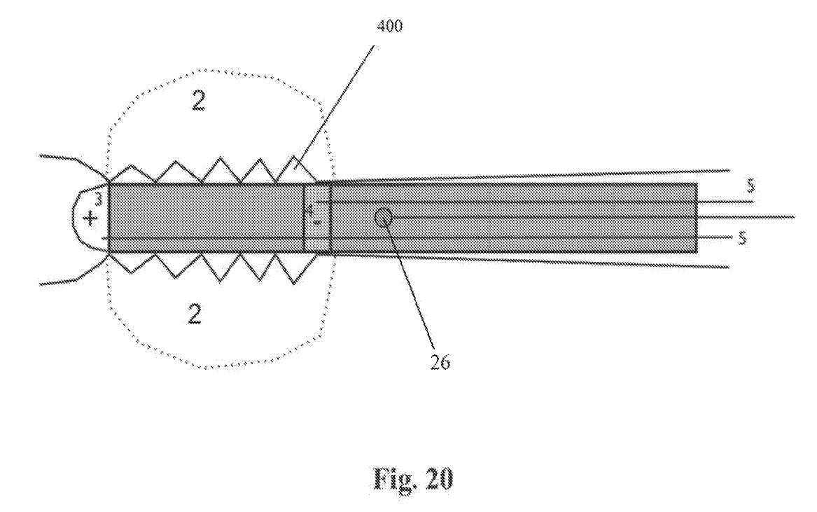

Each of the probes 20 described above also preferably comprises one or more EM sensors 26, such as those described above, on various portions of probe 20 to allow the position of the probe 20 and various parts thereof to be monitored and tracked in real time (see FIG. 20). Alternatively, instead of utilizing EM sensors, EMS treatment probes 20 may be tracked in real time and guided using endoscopy, ultrasound or other imaging means known in the art.

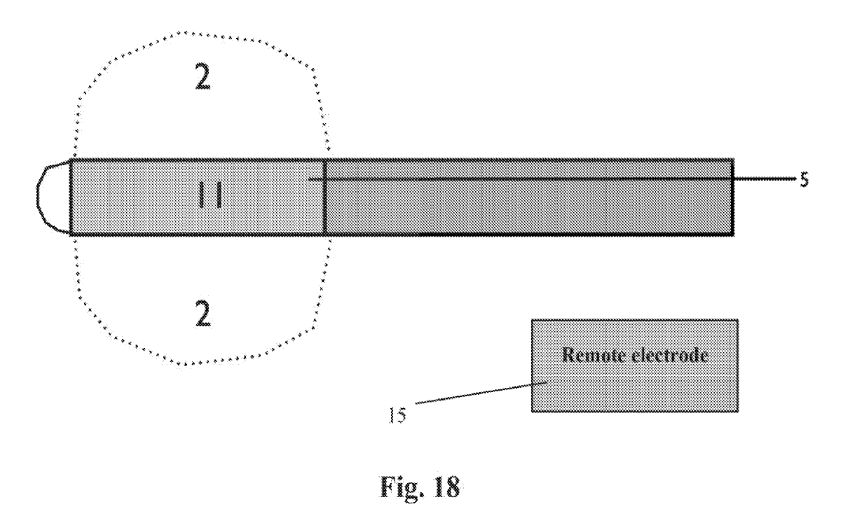

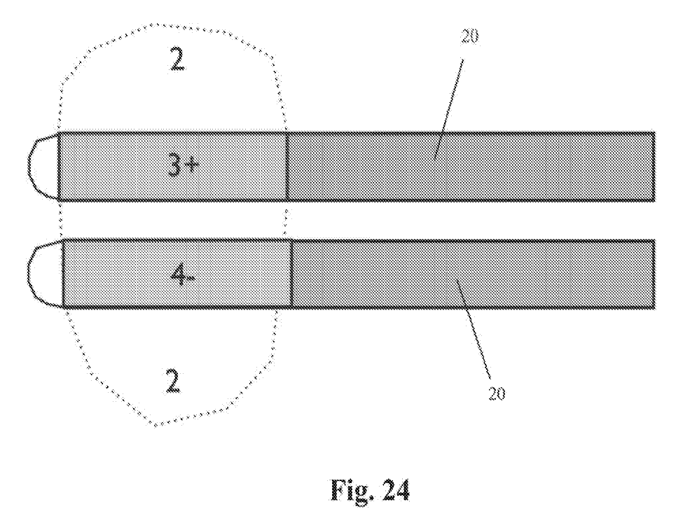

One of ordinary skill in the art will understand that the EMS treatment probe(s) 20 may take various forms provided that they are still capable of delivering EMS pulses from the EMB pulse generator 14 of the type, duration, etc. described above. For example, the EMB treatment probes 20 have been described herein as a rigid assembly, but may also be semi-rigid assembly with formable, pliable and/or deformable components. As another example, EMB treatment probes 20 may be unipolar 11 and used with an indifferent electrode placed on a remote location from the area of treatment (see FIG. 18). In yet another embodiment, two EMB treatment probes 20 may be used, wherein each probe has one each of a positive and negative electrode (See FIG. 24).

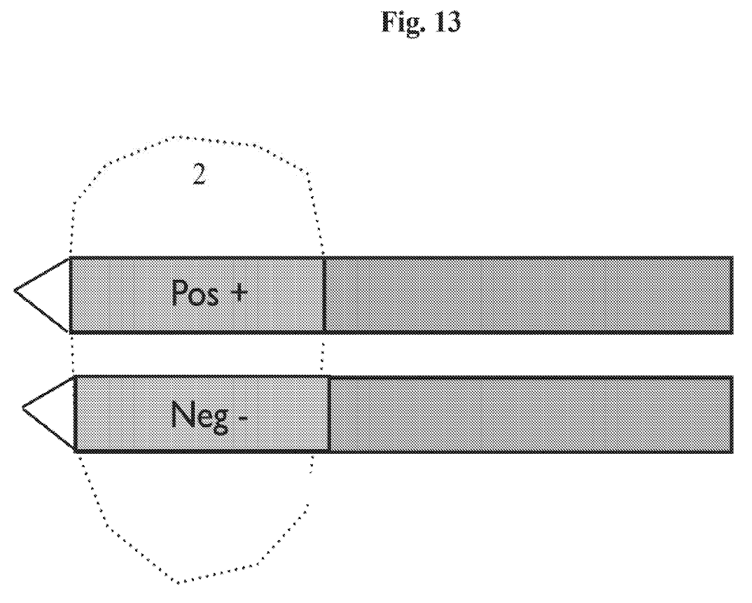

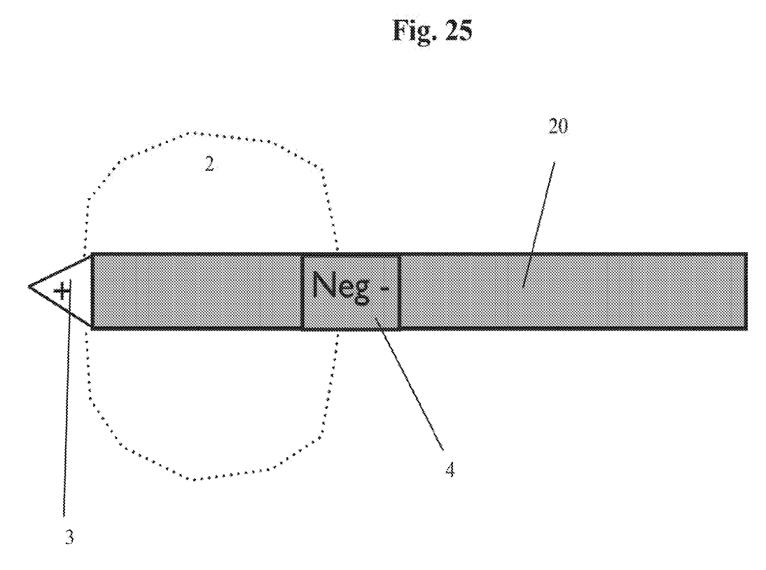

As yet another example, EMB probe 20 may comprise one or more sharp, rigid needles that can be placed interstitially from a transperineal route or a transrectal route. These may be used as pairs in a bipolar mode (see FIG. 13) with one probe 20 containing a positive electrode 3 and the other probe 20 containing a negative electrode 4, or both positive and negative electrodes 3, 4 placed on a single probe 20 (see FIG. 25). Alternatively, the needle probes 20 may be used in a monopolar mode with a unipolar electrode 11 placed on the probe 20 and an indifferent electrode 15 placed on the patient's body in a remote location (see FIG. 18). Rigid needle-type probes 20 may comprise one or more of each of the types of sensors/transmitters/features described above with reference to probes 20.

In yet another embodiment, the two curved, electrode-bearing needles 17 containing electrodes 3, 4 can be placed though a scope and be visualized as they extend out of the scope and, under direct scope visualization, pierce the walls of the urethra and extend into the BPH tissue for the treatment of prostate BPH (see FIG. 29).

Ultrasound Scanner 500

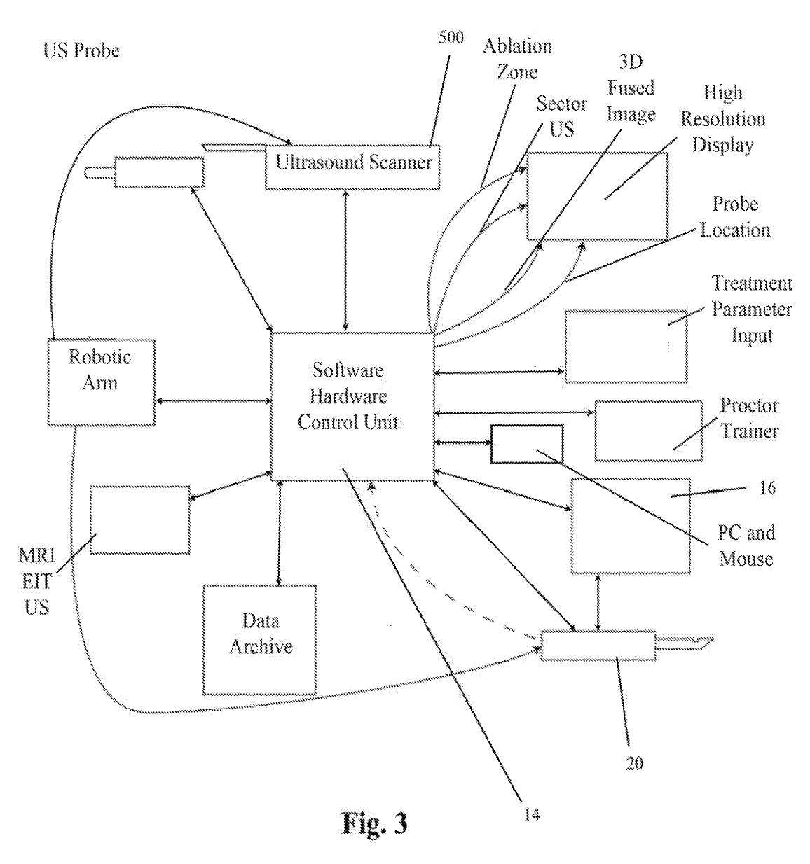

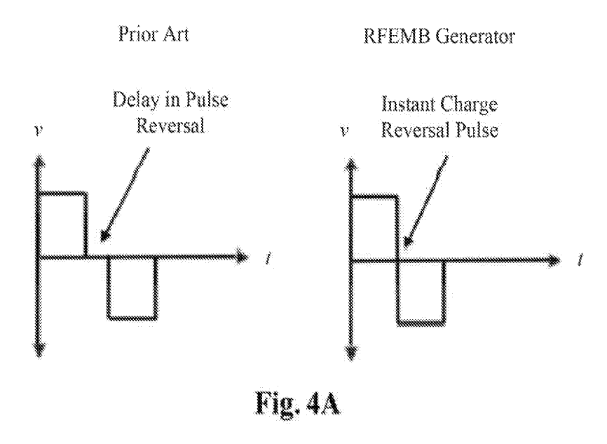

Unlike irreversible electroporation, electrical membrane breakdown EMB causes immediate visually observable tissue changes which can be monitored by an ultrasound scanner 500 (see FIG. 3) to show cellular membrane destruction and immediate cell death. As a result, the method of the present invention may include visual evaluation of the treated target tissue 2 via an ultrasound scanner 500 to verify treatment efficacy immediately upon completion of each tissue treatment during the ongoing therapy procedure, while the patient is still in position for additional, continued or further treatment. Preferably, ultrasound scanner 500 is operatively connected to SHCU 14 such that SHCU 14 may direct ultrasound scanner 500 to scan certain areas of the patient's body and/or receive images from ultrasound scanner 500 for display to the operator.

Additional treatment may be immediately administered via, i.e., EMB treatment probe 20, based on the information obtained from the sensors on the probe or visual determination of treatment efficacy through visual evaluation using ultrasound scanner 500, without renaming the treatment probe 20 from the treatment area 2.

Trackable Anesthesia Needles 300

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather anesthesia can be applied locally for the control of pain without the need for the deeper and riskier levels of sedation.

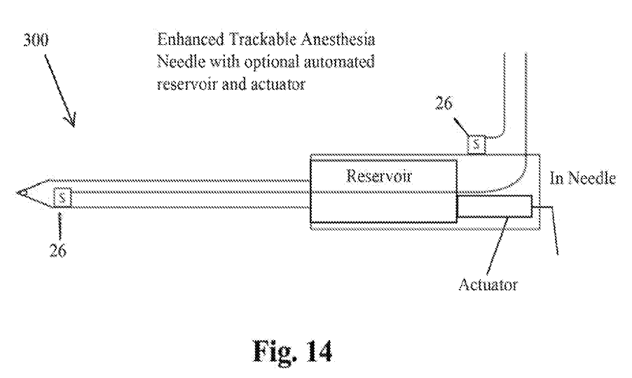

For this purpose, one or more trackable anesthesia needles 300 may be provided. With reference to FIG. 14, Anesthesia needles 300 may be of the type known in the art and capable of delivering anesthesia to the Neurovascular bundles or other potential treatment regions, including the point of entry of needle 300, EMB probe 20, or any of the other devices described herein through the skin to enhance pain relief. Anesthesia needles 300 may also comprise sensor/transmitters 26 (electromagnetic or otherwise) built into the needle and/or needle body to track the location anesthesia needle 300. Anesthesia needles 300 are preferably operatively connected to SHCU 14 to enable real-time tracking of anesthesia needle 300 by SHCU 14 and/or to monitor administration of anesthesia, as described in more detail below.

Alternatively, trackable anesthesia needles 300 may be omitted in favor of conventional anesthesia needles which may be applied by the physician using conventional manual targeting techniques and using the insertion point, insertion path and trajectories generated by the software according to the present invention, as described in further detail below.

Software Hardware Control Unit (SHCU) 14 and Treatment System Software

With reference to FIG. 3, the Software Hardware Control Unit (SHCU) 14 is operatively connected to one or more (and preferably all) of the therapeutic and/or diagnostic probes/needles, imaging devices and energy sources described herein: namely, in a preferred embodiment, the SUCH 14 is operatively connected to one or more EMB pulse generator(s) 16, EMB treatment probe(s) 20, ultrasound scanner 500 and trackable anesthesia needle(s) 300 via electrical/manual connections for providing power to the connected devices as necessary and via data connections, wired or wireless, for receiving data transmitted by the various sensors attached to each connected device. SHCU 14 is preferably operatively connected to each of the devices described herein such as to enable SHCU 14 to receive all available data regarding the operation and placement of each of these devices. For example, SHCU 14 may be connected to one or more trackable anesthesia needles 300 via a fluid pump through which liquid medication is provided to anesthesia needle 300 such that SHCU 14 may monitor and/or control the volume, rate, type, etc. of medication provided through needle(s) 300.

In an alternative embodiment, SHCU 14 is also connected to one or more of the devices herein via at least one robot arm such that SHCU 14 may itself direct the placement of various aspects of the device relative to a patient, potentially enabling fully automatized and robotic treatment of unwanted BPH tissue via EMB. It is envisioned that the system disclosed herein may be customizable with respect to the level of automation, i.e. the number and scope of components of the herein disclosed method that are performed automatically at the direction of the SHCU 14. At the opposite end of the spectrum from a fully automated system, SHCU 14 may operate software to guide a physician or other operator through a video monitor, audio cues, or some other means, through the steps of the procedure based on the software's determination of the best treatment protocol, such as by directing an operator where to place the EMB treatment probe 20, etc. As examples of semi-automation, SHCU 14 may be operatively connected to at least one robotic arm comprising an alignment tool capable of supporting a treatment probe 20, or providing an axis for alignment of probe 20, such that the tip of probe 20 is positioned at the correct point and angle at the surface of the patient's skin to provide a direct path along the longitudinal axis of probe 20 to the preferred location of the tip of probe 20 within the treatment area 2. In another embodiment, as described in more detail below, SHCU 14 provides audio or visual cues to the operator to indicate whether the insertion path of probe 20 is correct. In each of these variations and embodiments, the system, at the direction of SHCU 14, directs the planning, validation and verification of the Predicted Ablation Zone (to be described in more detail below), to control the application of therapeutic energy to the selected region so as to assure proper treatment, to prevent damage to sensitive structures and/or to provide tracking, storage, transmission and/or retrieval of data describing the treatment applied.

In a preferred embodiment, SHCU is a data processing system comprising at least one application server and at least one workstation comprising a monitor capable of displaying to the operator a still or video image, and at least one input device through which the operator may provide inputs to the system, i.e. via a keyboard/mouse or touch screen, which runs software programmed to control the system in two “modes” of operation, wherein each mode comprises instructions to direct the system to perform one or more novel features of the present invention. The software according to the present invention may preferably be operated from a personal computer connected to SHCU 14 via a direct, hardwire connection or via a communications network, such that remote operation of the system is possible. The two contemplated modes are Planning Mode and Treatment Mode. However, it will be understood to one of ordinary skill in the art that the software and/or operating, system may be designed differently while still achieving the same purposes. In all modes, the software can create, manipulate, and display to the user via a video monitor accurate, real-time three-dimensional images of the human body, which images can be zoomed, enlarged, rotated, animated, marked, segmented and referenced by the operator via the system's data input device(s). As described above, in various embodiments of the present invention the software and SHCU 14 can partially or fully control various attached components, probes, needles or devices to automate various functions of such components, probes, needles or devices, or facilitate robotic or remote control thereof.

Planning Mode

The SHCU is preferably operatively connected to one of more external imaging sources such as an magnetic resonance imaging (MRI), ultrasound (US), electrical impedance tomography (EIT), or any other imaging deice known in the art and capable of creating images of the human body. Using inputs from these external sources, the SHCU first creates one or more “3D Fused Images” of the patient's body in the region of the unwanted BPH tissue. The 3D Fused Images provide a 3D map of the selected treatment area 2 within the patient's body over which locational data obtained from the one or more probes, needles or ultrasound scans according to the present invention may be overlaid to allow the operator to plan and monitor the treatment in real-time against a visual of the actual treatment area 2.

In a first embodiment, a 3D Fused Image would be created from one or more MRI and ultrasound image(s) of the same area of the patient's body. An MRI image used for this purpose may comprise a multi-parametric magnetic resonance image created using, i.e., a 3.0 Telsa MRI scanner such as Achieva, manufactured by Philips Healthcare) with a 16-channel cardiac surface coil (such as a SENSE coil, manufactured by Philips Healthcare) placed over the pelvis of the patient with an endorectal coil (such as the BPX-30, manufactured by Medrad). MRI sequences obtained by this method preferably include: a tri-planar T2-weighted image, axial diffusion weighted imaging with apparent diffusion coefficient (ADC) mapping, 3-dimensional point resolved spatially localized spectroscopy, and an axial dynamic contrast enhanced MRI. An ultrasound image used for this purpose may be one or more 2D images obtained from a standard biplane transrectal ultrasound probe (such as the Hitachi EUB 350). The ultrasound image may be formed by, i.e., placing an EM field generator (such as that manufactured by Northern Digital Inc.) above the pelvis of the patient, which allows for real-time tracking of a custom ultrasound probe embedded with a passive EM tracking sensor (such as that manufactured by Traxtal, Inc.).

The 3D fused image is then formed by the software according to the present invention by encoding the ultrasound data using a position-encoded prostate ultrasound stepping device (such as that manufactured by Civco Inc) and then overlaying a virtual brachytherapy grid over the 3D ultrasound fused MRI image. A brachytherapy grid is positionally correlated to the resultant image by its fixed position to the US probe by the US stepping device. The software according to the present invention also records of the position of the suspected BPH tissue obtained as collected b ultrasound scans for later use in guiding therapy.

This protocol thus generates a baseline, diagnostic 3D Fused Image and displays the diagnostic 3D Fused Image to the operator in real time via the SHCU video monitor. Preferably, the system may request and/or receive additional 3D ultrasound images of the treatment area 2 during treatment and fuse those subsequent images with the baseline 3D Fused Image for display to the operator.

As an alternate means of creating the 3D Fused Image, a 2-dimensional US sweep of the treatment area 2 is performed in the axial plane to render a three-dimensional ultrasound image that is then registered and fused to a previously taken MRI using landmarks common to both the ultrasound image and MRI image such as the capsular margins of the prostate and urethra. Areas suspicious for causing BPH identified on MRI are semi-automatically superimposed on the real-time TRUS image. The 3D Fused Image as created by any one of the above methods is then stored in the non-transitive memory of the SHCU, which may employ additional software to locate and electronically tag within the 3D Fused Image specific areas in the prostate or its vicinity, including sensitive or critical structures and areas that require anesthesia such as the Neurovascular Bundles, i.e. to enable the guidance of standard or trackable anesthesia needles to those locations. The SHCU then displays the 3D Fused Image to the operator alone or overlaid with locational data from each of the additional devices described herein where available. The 3D Fused Image may be presented in real time in sector view, or the software may be programmed to provide other views based on design preference. As described above, the software may then direct the operator and/or a robotic arm to take a further ultrasound scan of the identified area of concern for unwanted BPH tissue, or in a specific location of concern based on an automated analysis of the imaging data and record the results of same, which additional imaging scan may be tracked in real time. Analysis of the image scan results which may be done by the system using automated image analysis capabilities, or a physician/technician, will indicate whether the tissue should be targeted for ablation. Thus, a 3D map of BPH targeted tissue in the area of concern within the patient's body may be created in this way. The software may employ an algorithm to determine where individual tissue areas should be evaluated further to ensure that all areas of concern in the region have been located evaluated, and indexed against the 3D Fused Image.

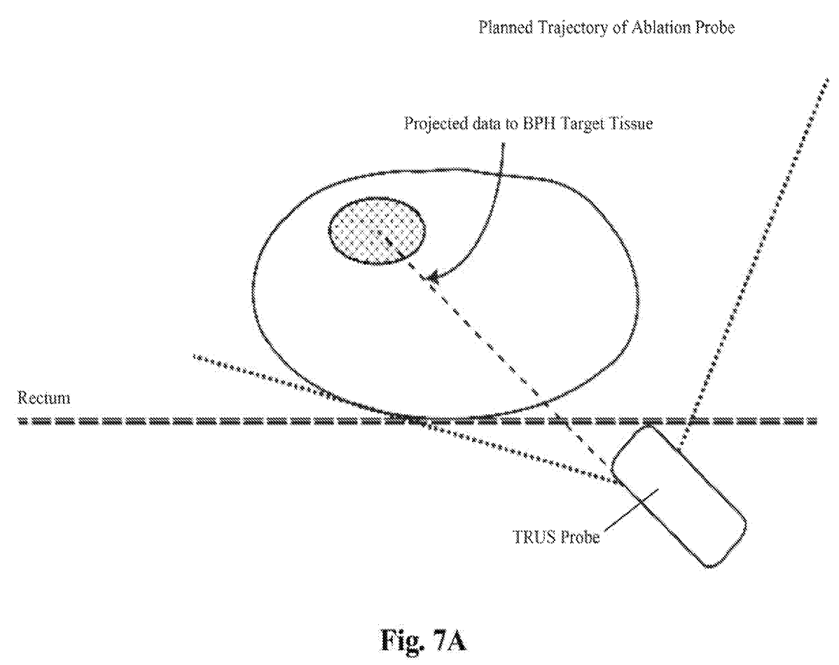

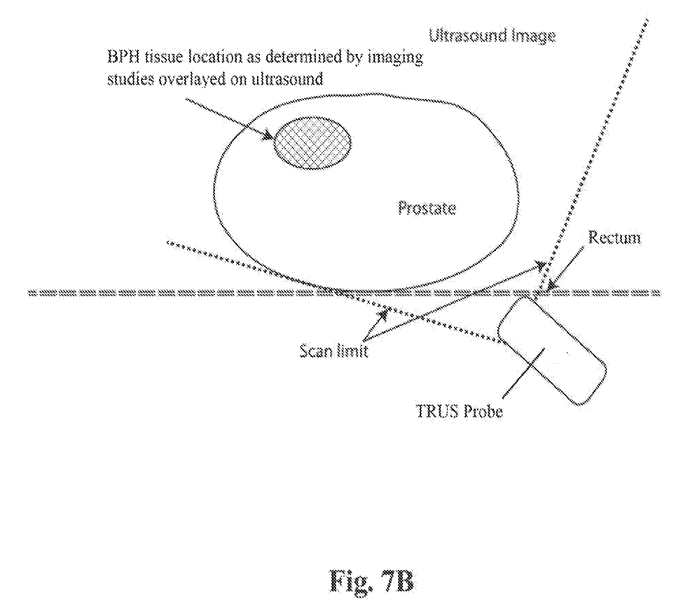

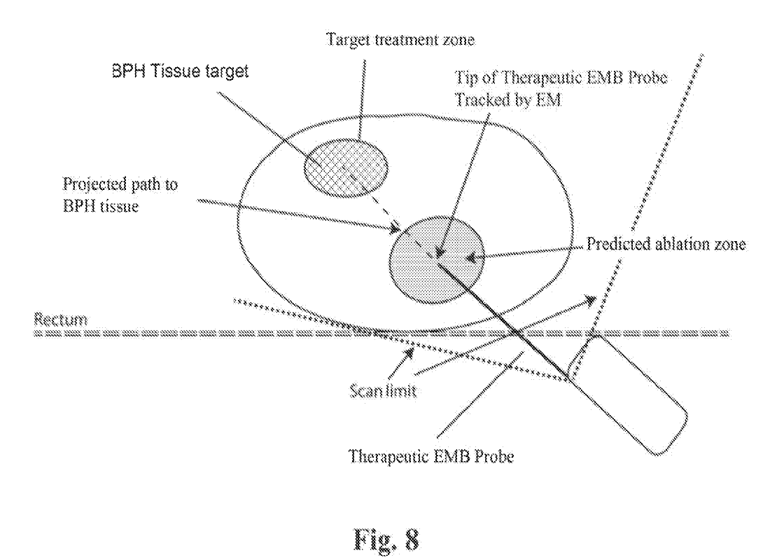

Using the image evaluation result data in conjunction with the 3D Fused Image, the software can create a targeted “3D Fused Image”, Which can be used as the basis for an office based treatment procedure for the patient (see FIGS. 7A-7B). The SHCU also preferably stores the image scan information indeed to location, orientation and scan number, which information can be provided to a radiologist for consultation if desired, or other treatment provider via a communications network to be displayed on his or her remote workstation, allowing the other treatment provider to interact with and record their findings or analysis about each image in real time.

Upon generation of one or more 3D Fused Images of the planned treatment area 2 and, preferably completion of the analysis of all of the image scans of the affected area, the SHCU may display to the operator via a video terminal the precise location(s) of one or more areas in the prostate (or other treatment area), or its vicinity, which require therapy, via annotations or markers on the 3D Fused Image(s): this area requiring therapy is termed the Target Treatment Zone. This information is then used by the system or by a physician to determine optimal placement of the EMB treatment probe(s) 20. Importantly, the 3D Fused Image should also contain indicia to mark Neurovascular Bundles (NVB), the location of which will be used to calculate a path for placement of one or more anesthesia needles for delivery of local anesthesia to the treatment area 2. If necessary due to changes in gland size, the geographic location of each marker can be revised and repositioned, and the 3D Fused Image updated in real time by the software, using 3D ultrasound data as described above. The system may employ an algorithm for detecting changes in gland size and requesting additional ultrasound scans, may request ultrasound scans on a regular basis, or the like.

In a preferred embodiment, the software may provide one or more “virtual” EMB treatment probes 20 which may be overlaid onto the 3D Fused Image by the software or by the treatment provider to determine the extent of ablation that would be accomplished with each configuration. The virtual probes also define a path to the target point by extending a line or path from the target point to a second point defining the entry point on the skin surface of the patient for insertion of the real EMB treatment probe. Preferably, the software is configured to test several possible probe 20 placements and calculate the probable results of treatment to the affected area via such a probe 20 (the Predicted Ablation Zone) placement using a database of known outcomes from various EMB treatment protocols or by utilizing an algorithm which receives as inputs various treatment parameters such as pulse number, amplitude, pulse width and frequency. By comparing the outcomes of these possible probe locations to the targeted BPH tissue volume as indicated by the 3D Fused Image, the system may determine the optimal probe 20 placement. Alternatively, the system may be configured to receive inputs from a physician to allow him or her to manually arrange and adjust the virtual EMB treatment probes to adequately cover the treatment area 2 and volume based on his or her expertise. The system may utilize virtual anesthesia needles in the same way to plan treatment.

When the physician is satisfied with the Predicted Ablation Zone coverage shown on the Target Treatment Zone based on the placement and configuration of the virtual EMB treatment probes and the virtual anesthesia needles, as determined by the system or by the physician himself, the physician “confirms” in the system (i.e. “locks in”) the three-dimensional placement and energy/medication delivery configuration of the grouping of virtual EMB treatment probes and virtual anesthesia needles, and the system registers the position of each as an actual software target to be over on the 3D Fused Image and used by the system for guiding the insertion of the real probe(s) and needle(s) according to the present invention (which may be done automatically by the system via robotic arms or by the physician by tracking his or her progress on the 3D Fused Image.

If necessary, EMB treatment, as described in further detail below, may be carried out immediately after the treatment planning of the patient is performed. Alternately, EMB treatment may take place days or even weeks after one or more diagnostic scanning and imaging studies are performed. In the latter case, the steps described with respect to the Planning Mode, above, may be undertaken by the software/physician at any point between diagnostic scanning and imaging and treatment.

Treatment Mode

The software displays, via the SHCU video monitor, the previously confirmed and “locked in” Target Treatment Zone, and Predicted Ablation Zone, with the location and configuration of all previously confirmed virtual probes/needles and their calculated insertion points, angular 3D geometry, and insertion depths, which can be updated as needed at time of treatment to reflect any required changes as described above.

Using the planned locations and targets established for the delivery of anesthesia, and the displayed insertions paths, the software then guides the physician (or robotic arm) in real time to place one or more anesthesia needles and then to deliver the appropriate amount of anesthesia to the targeted locations (i.e., in the vicinity of the Neurovascular Bundles). Deviations from the insertion path previously determined by the system in relation to the virtual needles/probes may be highlighted by the software in real time so as to allow correction of targeting at the earliest possible time in the process. This same process allows the planning and placement of local anesthesia needles as previously described. In some embodiments, the system may employ an algorithm to calculate the required amount of anesthesia based on inputs such as the mass of the tissue to be treated and individual characteristics of the patient which may be inputted to the system manually by the operator or obtained from a central patient database via a communications network, etc.

Once anesthesia has been administered, the system displays the Predicted Ablation Zone and the boundaries thereof as an overlay on the 3D Fused Image including the Target Treatment Zone and directs the physician (or robotic arm) as to the placement of each EMB treatment probe 20. The Predicted Ablation Zone may be updated and displayed in real time as the physician positions each probe 20 to give graphic verification of the boundaries of the Target Treatment Zone, allowing the physician to adjust and readjust the positioning of the Therapeutic EMB Probes, sheaths, electrode exposure and other treatment parameters (which in turn are used to update the Predicted Ablation Zone). When the physician (or, in the case of a fully automated system, the software) is confident of accurate placement of the probes, he or she may provide such an input to the system, which then directs the administration of EMB pulses via the EMB pulse generator 16 and probes 20.

The SHCU controls the pulse amplitude 30, frequency 31, polarity and shape provided by the EMB pulse generator 16, as well as the number of pulses 32 to be applied in the treatment series or pulse train, the duration of each pulse 32, and the inter pulse burst delay 33. Although only two are depicted in FIG. 10 due to space constraints, EMB ablation is preferably performed by application of a series of not less than 100 electric pulses 32 in a pulse train so as to impart the energy necessary on the target tissue 2% without developing thermal issues in any clinically significant way. The width of each individual pulse 32 is preferably from 100 to 1000 μs with an inter pulse burst interval 33 during which no voltage is applied in order to facilitate heat dissipation and avoid thermal effects. The relationship between the duration of each pulse 32 and the frequency 31 (period) determines the number of instantaneous charge reversals experienced by the cell membrane during each pulse 32. The duration of each inter pulse burst interval 33 is determined by the controller 14 based on thermal considerations. In an alternate embodiment the system is further provided with a temperature probe 22 inserted proximal to the target tissue 2 to provide a localized temperature reading at the treatment site to the SHCU 14. The temperature probe 22 may be a separate, needle type probe having a thermocouple tip, or may be integrally formed with or deployed from one or more of the needle electrodes, or the Therapeutic EMB Probes. The system may further employ an algorithm to determine proper placement of this probe for accurate readings from same. With temperature feedback in real time, the system can modulate treatment parameters to eliminate thermal effects as desired by comparing the observed temperature with various temperature set points stored in memory. More specifically, the system can shorten or increase the duration of each pulse 32 to maintain a set temperature at the treatment site to, for example, create a heating (high temp) for the needle tract to prevent bleeding or to limit heating (low temp) to prevent any coagulative necrosis. The duration of the inter pulse burst interval can be modulated in the same manner in order to eliminate the need to stop treatment and maximizing the deposition of energy to accomplish EMB. Pulse amplitude 30 and total number of pulses in the pulse train may also be modulated for the same purpose and result.

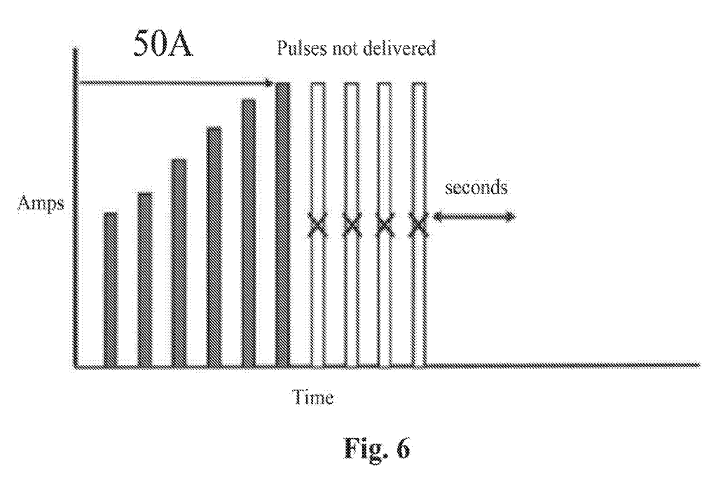

In yet another embodiment, the SHCU may monitor or determine current flow through the tissue during treatment for the purpose of avoiding overheating while yet permitting treatment to continue by reducing the applied voltage. Reduction in tissue impedance during treatment due to charge buildup and membrane rupture can cause increased current flow which engenders additional heating at the treatment site. With reference to FIG. 6, prior treatment methods have suffered from a need to cease treatment when the current exceeds a maximum allowable such that treatment goals are not met. As with direct temperature monitoring, the present invention can avoid the need to stop treatment by reducing the applied voltage and thus current through the tissue to control and prevent undesirable clinically significant thermal effects. Modulation of pulse duration and pulse burst interval duration may also be employed by the controller 14 for this purpose as described.

During treatment, the software captures all of the treatment parameters, all of the tracking data and representational data in the Predicted Ablation Zone, the Target Treatment Zone and in the 3D Mapped Image as updated in real time to the moment of therapeutic trigger. Based on the data received by the system during treatment, the treatment protocol may be adjusted or repeated as necessary.

The software may also store, transmit and/or forwarding treatment data to a central database located on premises in the physician's office and/or externally via a communications network so as to facilitate the permanent archiving and retrieval of all procedure related data. This will facilitate the use and review of treatment data, including for diagnostic purposes for treatment review purposes and other proper legal purposes including regulatory review.

The software may also transmit treatment data in real time to a remote proctor/trainer who can interact in real time with the treating physician and all of the images displayed on the screen, so as to insure a safe learning experience for an inexperienced treating physician, and so as to archive data useful to the training process and so as to provide system generated guidance for the treating physician. In another embodiment, the remote proctor can control robotically all functions of the system.

In other embodiments of the present invention, some or all of the treatment protocol may be completed by robotic arms, which may include an ablation probe guide which places the specially designed Therapeutic EMB Probe (or an ordinary ablation probe but with limitations imposed by its design) in the correct trajectory to the tumor. Robotic arms may also be used to hold the US transducer in place and rotate it to capture images for a 3D US reconstruction. Robotic arms can be attached to an anesthesia needle guide which places the anesthesia needle in the correct trajectory to the Neurovascular Bundles to guide the delivery of anesthesia by the physician.

In other embodiments, the robotic arm can hold the anesthesia needle itself or a trackable anesthesia needle (see FIG. 14) with sensor-transmitters and actuators built in, that can be tracked in real time, and that can feed data to the software to assure accurate placement thereof and enable the safe, accurate and effective delivery of anesthesia to the Neurovascular bundles and other regions, and can directly insert the needle into the targeted areas of the Neurovascular Bundle and other regions using and reacting robotically to real time positioning data supported by the 3D Fused Image and Predicted Ablation Zone data and thereby achieving full placement robotically, and upon activation of the flow actuators, the delivery of anesthesia as planned or confirmed by the physician.

In addition, the robotic arm can hold the Therapeutic EMB Probe itself and can directly insert the probe into the patient's tumor using, and reacting robotically to real time positioning data supported by the 3D Fused Image and Predicted Ablation Zone data and thereby achieving full placement robotically.

Robotic components capable of being used for these purposes include the iSR'obot™ Mona Lisa robot, manufactured by Biobot Surgical Pte. Ltd. In such embodiments the Software supports industry standard robotic control and programming languages such as RAIL, AML, VAL, AL, RPL, PYRO, Robotic Toolbox for MATLAB and OPRoS as well as other robot manufacturer's proprietary languages.

The SHCU can fully support Interactive Automated Robotic Control through a proprietary process for image sub-segmentation of prostate structures for planning and performing robotically guided therapeutic interventions in an office based setting.

Sub-segmentation is the process of capturing and storing precise image detail of the location size and placement geometry of the described object so as to be able to define, track, manipulate and display the object and particularly its three-dimensional boundaries and accurate location in the body relative to the rest of the objects in the field and to the anatomical registration of the patient in the system so as to enable accurate three-dimensional targeting of the object or any part thereof, as well as the three-dimensional location of its boundaries in relation to the locations of all other subsegmented objects and computed software targets and needle and probe pathways. The software sub-segments out various critical prostate substructures, such as the neuro-vascular bundles, peripheral zone, ejaculatory ducts, urethra, rectum, and Denonvilliers Fascia in a systematic and programmatically supported and required fashion, which is purposefully designed to provide and enable the component capabilities of the software as described herein.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

### STATEMENT OF INDUSTRIAL APPLICABILITY

Benign prostatic hyperplasia (BPH) is a disease affecting nearly eighty percent of men by the age of eighty, and characterized by distressing urination symptoms which can negatively affect the quality of life of affected men. Left untreated, BPH can cause even more severe complications, such as urinary tract infection, acute urinary retention, and uremia. The known treatments for BPH are often painful, embarrassing, comprising long treatment or healing times and/or unknown side effects. There would be great industrial applicability in an effective ablation of BPH tissue that was minimally invasive and less traumatic than classic methods of removing unwanted tissue by surgical excision, and which could be conducted without the need for general anesthesia, which may have dangerous side effects. The instant invention fulfills this need by utilizing Radio-Frequency Electrical Membrane Breakdown to destroy the cellular membranes BPH tissue without denaturing the intra-cellular contents of the cells comprising the tissue.

We claim:

1. A method of ablating undesirable soft tissue in a living subject using radio frequency electrical membrane breakdown, the method comprising:
identifying a location of said soft tissue within said subject;
introducing at least one electrode to said location within said subject; and
applying to said soft tissue at said location, via said at least one electrode, an electric field sufficient to cause electrical membrane breakdown of a cell membrane of a plurality of cells of said soft tissue to cause immediate spillage of all intracellular components into an extracellular space and exposure of an internal constituent part of said cell membrane to said extracellular space;
wherein said undesirable soft tissue comprises benign prostatic hyperplasia tissue.

2. The method of claim 1, wherein said step of introducing at least one electrode to said location comprises:
taking a 3D image of said location;
inserting one or more trackable biopsy needles into said location;
collecting one or more data points from said one or more trackable biopsy needles inserted into said location;
overlaying said one or more data points from said one or more trackable biopsy needles onto said 3D image of said location to form a 3D fused image of said location;
using said 3D fused image of said location to determine one or more insertion points on said subject for one or more therapeutic EMB probes; and
inserting said one or more therapeutic EMB probes through said one or more insertion points on said subject, said one or more therapeutic EMB probes each containing one or more of said at least one electrodes.

3. The method of claim 2, wherein said step of using said 3D fused image of said location to determine one or more insertion points on said subject comprises:
generating one or more virtual probe configurations, each of said one or more virtual probe configurations comprising the virtual locations of one or more virtual EMB treatment probes proximate said location;
overlaying said one or more virtual probe configurations onto said 3D fused image;
determining the extent of ablation that would be accomplished with each of said one or more virtual probe configurations to generate a predicted ablation outcome for each of said one or more virtual probe configurations; and
determining said one or more insertion points by comparing said one or more predicted ablation outcomes of each of said one virtual probe configurations.

4. The method of claim 1, wherein said step of introducing at least one electrode to said location comprises:
performing an MRI on said location to generate an MRI image;
performing a two-dimensional US sweep of said location in an axial plane thereof to generate a three-dimensional ultrasound image;
registering said three-dimensional ultrasound image to said MRI image using landmarks common to both said three-dimensional ultrasound image and said MRI image;
identifying one or more areas suspicious for causing BPH on said MRI image;
semi-automatically superimposing said one or more areas over a real-time TRUS image of said location to generate a superimposed image of said location;
using said superimposed image of said location to determine one or more insertion points on said subject for one or more therapeutic EMB probes; and
inserting said one or more therapeutic EMB probes through said one or more insertion points on said subject, said one or more therapeutic EMB probes each containing one or more of said at least one electrodes.

5. The method of claim 4, wherein said step of using said superimposed image of said location to determine one or more insertion points on said subject comprises:
generating one or more virtual probe configurations, each of said one or more virtual probe configurations comprising the virtual locations of one or more virtual EMB treatment probes proximate said location;
overlaying said one or more virtual probe configurations onto said superimposed image;
determining the extent of ablation that would be accomplished with each of said one or more virtual probe configurations to generate a predicted ablation outcome for each of said one or more virtual probe configurations; and
determining said one or more insertion points by comparing said one or more predicted ablation outcomes of each of said one or more virtual probe configurations.

6. The method of claim 2, wherein said steps of inserting one or more trackable biopsy needles into said location and inserting said one or more therapeutic EMB probes through said one or more insertion points on said subject are conducted at least in part by a robotic arm.

7. The method of claim 1, wherein said step of introducing said at least one electrode comprises:
inserting a catheter through a urethra of said living subject; and
inserting one or more therapeutic EMB probes through a lumen of said catheter, wherein said one or more therapeutic EMB probes each contain one or more of said at least one electrodes.

8. The method of claim 7, wherein following said step of inserting said catheter through stud urethra of said living subject, said catheter is held in the neck of a bladder of said living subject by a friction fit of a balloon on a distal end of said catheter.

9. The method of claim 7, further comprising inserting one or more stents into said urethra of said living subject using said catheter.

10. The method in claim 1, wherein the method is monitored by endoscopic ultrasound.

11. The method of claim 1, wherein the method is carried out without the application of general anesthesia or a neuromuscular blockade to said living subject.

12. A system for ablating benign prostatic hyperplasia tissue in a living subject using radio frequency electrical membrane breakdown, the system comprising:
at least one EMB pulse generator capable of generating an electric field sufficient to cause electrical membrane breakdown of a cell membrane of a plurality of cells of said benign prostatic hyperplasia tissue to cause immediate spillage of all intracellular components into an extracellular space and exposure of an internal constituent part of said cell membrane to said extracellular space;
at least one EMB treatment probe capable of delivering said electric field to said soft tissue;
at least one ultrasound scanner; and
at least one software hardware control unit operatively connected to said at least one EMB pulse generator and said at least one EMB treatment probe.

13. The system of claim 12, wherein said at least one EMB treatment probe comprises:
a core comprised of an electrically conductive material;
an outer electrode covering said core on at least one side; and
an insulating sheath comprised of a non-electrically-conductive material, said insulating sheath forming a barrier between said core and said outer electrode.

14. The system of claim 13, wherein said outer electrode is mounted on said insulating sheath, and wherein said outer electrode and said insulating sheath are movable as a unit along a lateral dimension of said core to enable adjustment of the lateral distance between a distal end of said core and said outer electrode.

15. The system of claim 14, further comprising at least one electromagnetic sensor on each of said core and said outer electrode.

16. The system of claim 13, wherein said at least one EMB treatment probe comprises at least one sensor capable of determining or quantifying cell death in tissue adjacent to said at least one sensor.

17. The system of claim 13, wherein said at least one EMB treatment probe comprises a hollow interior defined by an inner lumen of sufficient diameter to accommodate a needle of one or more standard gauges.

18. The system of claim 17, wherein said at least one EMB treatment probe comprises an outer electrode on an outer surface thereof, and further comprising a needle sized to fit within said inner lumen of said EMB treatment probe, said needle comprising a needle electrode on a distal end thereof, wherein a polarity of said needle electrode is not equal to a polarity of said outer electrode.

19. The system of claim 13, wherein said at least one EMB treatment probe comprises an expandable balloon at a distal end thereof, said expandable balloon further comprising one or more electrodes for delivering said electric field.

20. The system of claim 13, wherein said at least one EMB treatment probe is a catheter-type probe, wherein said at least one EMB treatment probe further comprises:
a central lumen;
a positive electrode disposed at a first location om an outer surface of said EMB treatment probe; and
a negative electrode disposed on at a second location on an outer surface of said EMB treatment probe, said first location and said second location being separated along a longitudinal dimension of said at least one EMB treatment probe.

21. The system of claim 20, wherein one of said positive electrode or said negative electrode is disposed on the end of an insulating sheath comprised of a non-electrically-conductive material, said insulating sheath being movable along a longitudinal axis of said at least one EMB treatment probe.

22. The method of claim 13, wherein said at least one EMB treatment probe further comprises one or more stents sized to fit within the urethra of said living subject.

23. The method of claim 22, wherein said one or more stents each comprise conducting and non-conducting areas corresponding to said at least one electrode.
